SEQUENCE LISTING (26 sequences)

SEQ ID NO: 1 - DNA, 22 bp, synthetic construct
  source 1..22: mol_type = other DNA
ttgattcagagcctcccattag

SEQ ID NO: 2 - DNA, 21 bp, synthetic construct
  source 1..21: mol_type = other DNA
gcgaggagtaggcttgtattt

SEQ ID NO: 3 - DNA, 20 bp, synthetic construct
  source 1..20: mol_type = other DNA
ctcagcaatcactggcatca

SEQ ID NO: 4 - DNA, 22 bp, synthetic construct
  source 1..22: mol_type = other DNA
actgggcaactccagaaataag

SEQ ID NO: 5 - DNA, 23 bp, synthetic construct
  source 1..23: mol_type = other DNA
catgtgcattctcttccaaagtc

SEQ ID NO: 6 - DNA, 20 bp, synthetic construct
  source 1..20: mol_type = other DNA
ctccttggtgaccgcattat

SEQ ID NO: 7 - DNA, 21 bp, synthetic construct
  source 1..21: mol_type = other DNA
agacgacaatgcagactaacc

SEQ ID NO: 8 - DNA, 20 bp, synthetic construct
  source 1..20: mol_type = other DNA
ctgcagcaatcccacatttc

SEQ ID NO: 9 - DNA, 20 bp, synthetic construct
  source 1..20: mol_type = other DNA
ggcgaaagtcagcctattga

SEQ ID NO: 10 - DNA, 20 bp, synthetic construct
  source 1..20: mol_type = other DNA
aggtgggaagatgaggaaga

SEQ ID NO: 11 - DNA, 22 bp, synthetic construct
  source 1..22: mol_type = other DNA
gctactgggtaatggtgaagaa

SEQ ID NO: 12 - DNA, 20 bp, synthetic construct
  source 1..20: mol_type = other DNA
gctgtgccagaactgtagaa

SEQ ID NO: 13 - DNA, 21 bp, synthetic construct
  source 1..21: mol_type = other DNA
ggaagatgtcaggatggttca

SEQ ID NO: 14 - DNA, 22 bp, synthetic construct
  source 1..22: mol_type = other DNA
cgaaggacaaattgaggcaaag

SEQ ID NO: 15 - DNA, 21 bp, synthetic construct
  source 1..21: mol_type = other DNA
cacagctggagggaagtaaat

SEQ ID NO: 16 - DNA, 23 bp, synthetic construct
  source 1..23: mol_type = other DNA
ttgagtaaggaagtgaggttgag

SEQ ID NO: 17 - DNA, 21 bp, synthetic construct
  source 1..21: mol_type = other DNA
gacagcctatgccaacaagta

SEQ ID NO: 18 - DNA, 21 bp, synthetic construct
  source 1..21: mol_type = other DNA
ttacaggaagggcaactcatc

SEQ ID NO: 19 - DNA, 22 bp, synthetic construct
  source 1..22: mol_type = other DNA
cgtcagccagtttgactatgta

SEQ ID NO: 20 - DNA, 22 bp, synthetic construct
  source 1..22: mol_type = other DNA
ctctcaaagaagctgaggatgg

SEQ ID NO: 21 - DNA, 20 bp, synthetic construct
  source 1..20: mol_type = other DNA
ccagcaatgccagcatattg

SEQ ID NO: 22 - DNA, 22 bp, synthetic construct
  source 1..22: mol_type = other DNA
cggtttctccttaccacttctt

SEQ ID NO: 23 - DNA, 23 bp, synthetic construct
  source 1..23: mol_type = other DNA
ccttggaggtttcttcacctatt

SEQ ID NO: 24 - DNA, 23 bp, synthetic construct
  source 1..23: mol_type = other DNA
ggtcatcccactgaagtctaatc

SEQ ID NO: 25 - DNA, 22 bp, synthetic construct
  source 1..22: mol_type = other DNA
gcaagacgaactaaagcgaaag

SEQ ID NO: 26 - DNA, 20 bp, synthetic construct
  source 1..20: mol_type = other DNA
tcggaactacgacggtatct

(19) United States
(12) Patent Application Publication
(10) Pub. No.: US 2023/0364051 A1
(43) Pub. Date: Nov. 16, 2023

(54) FORMULATIONS AND METHODS FOR IMPROVING AVIAN HEALTH

(71) Applicant: CORNELL UNIVERSITY, Ithaca, NY (US)

(72) Inventors: Elad Tako, Berkshire, NY (US); Jacquelyn Cheng, Ithaca, NY (US)

(21) Appl. No.: 18/194,867

(22) Filed: Apr. 3, 2023

Related U.S. Application Data
(60) Provisional application No. 63/326,329, filed on Apr. 1, 2022.

(51) Int. Cl. A61K 31/352; A23K 20/121; A61P 3/02; A23K 50/75; A23K 10/30; A23K 50/60

(52) U.S. Cl. CPC A61K 31/352; A23K 10/30; A23K 20/121; A23K 50/60; A23K 50/75; A61P 3/02

(57) ABSTRACT

Described in several example embodiments herein are formulations containing an amount of genistein optionally effective, in an avian, to modify the avian intestinal microbiome, increase a nutrient and/or mineral bioavailability and/or improve nutrient and/or mineral status in the avian, increase body weight gain, decrease feed conversion ratio, correct an iron deficiency, correct a Zn deficiency, support normal avian intestinal health and/or functionality and/or improve intestinal health and/or functionality, or any combination thereof and methods of the same.

### CROSS-REFERENCE TO RELATED APPLICATIONS

This application claims the benefit of and priority to U.S. Provisional Pat. Application No. 63/326,329, filed on Apr. 1, 2022, the contents of which is incorporated by reference herein in its entirety.

### SEQUENCE LISTING

This application contains a sequence listing filed in electronic form as an xml file entitled “CORNL-0785US_ST26.xml”, created on Mar. 30, 2023, and having a size of 23,758 bytes. The content of the sequence listing is incorporated herein in its entirety.

### TECHNICAL FIELD

The subject matter disclosed herein is generally directed to formulations and methods for improving avian health, and more particularly, formulations and methods of administering genistein for improving poultry health.

### BACKGROUND

The use of antimicrobials in various livestock industries, including poultry, to prevent and treat disease has been under increasing scrutiny over the past few decades for their role in promoting antimicrobial resistance in key animal and human pathogens. Indeed, over the past several decades poultry producers have faced the implementation of strict government guidelines drastically limiting the use of antimicrobials in production. Several prominent classes of antibiotics, e.g., fluoroquinolones and cephalosporins, important in human medicine have been phased out of use in poultry production. Given the continued pressure to reduce the use of antimicrobials for food production, particularly as growth promoters by preventing sub clinical levels of infection, there exists a need for alternatives to antimicrobials for poultry and livestock production.

Citation or identification of any document in this application is not an admission that such a document is available as prior art to the present invention.

### SUMMARY

Described in certain example embodiments are methods of modifying an intestinal microbiome structure of an avian comprising administering, to an avian, an amount of genistein or a genistein formulation, optionally a dietary supplement, feed supplement, or feed formulation, wherein the amount of genistein or genistein formulation is effective to (a) modify the intestinal microbiome structure of the avian (e.g., modify to increase the number of probiotic, short chain fatty acid (SCFA) producing bacteria, and/or non-pathogenic bacteria (e.g., Bifidobacterium, lactobacillus, L. plantarum, Clostridium bacteria or any combination thereof) and/or decrease the number of pathogenic or non-probiotic bacterium (e.g., E. coli); (b) increase a nutrient and/or mineral bioavailability and/or improve nutrient and/or mineral status in the avian; (c) increase body weight gain of the avian; (d) decrease feed conversion ratio of the avian; (e) support normal avian intestinal health and/or functionality and/or improve intestinal health and/or functionality as optionally measured by intestinal morphology, (including goblet cell number, crypt Paneth cell number), microbiome structure, intestinal health or functionality expression signature (intestinal nutrient transporter expression, intestinal mineral transporter expression, intestinal inflammatory marker expression, intestinal NaK/ATPase exchanger expression, expression of a mineral regulator in the intestine or liver, or any combination thereof), and any combination thereof; (f) correct an iron or Zn deficiency in the avian; (g) modify expression of one or more of the following genes in the avian: DMT1, Ferroportin, DcytB, Bepcidin, Δ-6-desaturase, ZIP6, ZnT7, NF-κβ, TNF-α, AP, SI, NaK/ATPase, and 18S; or (h) any combination thereof.

Described in certain example embodiments, are methods of preventing intestinal abnormality, dysbiosis, malabsorption, malnutrition, infection, and/or disease in an avian, the method comprising administering, to the avian, an amount of genistein or a genistein formulation, optionally a dietary supplement, feed supplement, or feed formulation, wherein the amount of genistein or genistein formulation is effective to preventing intestinal abnormality, dysbiosis, malabsorption, malnutrition, infection, and/or disease in the avian.

In certain example embodiments, the amount of genistein administered ranges from about 0.1 wt. % to 5 wt. %, optionally about 0.1 wt. % to 1 wt. %, about 1 wt. % to about 5 wt. %, or about 1.25 wt. % to about 2.5 wt. % of the genistein formulation, of a dietary supplement, of a feed supplement, of a total diet, or of a feed formulation or is otherwise administered at an amount such that the total amount of genistein in a total diet of the avian ranges from about 0.1 wt. % to 5 wt. %, optionally about 0.1 wt. % to 1 wt. %, about 1 wt. % to about 5 wt. %, or about 1.25 wt. % to about 2.5 wt. %.

In certain example embodiments, the avian is a developing embryo.

In certain example embodiments, the avian is a neonate, a young avian, an adolescent avian, or a mature adult avian. In certain example embodiments, administration is during embryonic development, optionally directly to a developing embryo or indirectly to a sexually mature avian.

In certain example embodiments, administration is via in ovo injection or intraamniotic injection. In certain example embodiments, administration is oral.

In certain example embodiments, the avian, is an avian of the genus Gallus, Melegris, Dromaius, Struthio, Phoenicopterus, Callipepla, Cyrtonyx, Dactylortyx, Philortyx, Colinus, Odontophorus, Oreortyx, Rhynchortyx, Coturnix, Synoicus, Perdicula, or Ophrysia. In certain example embodiments, the avian is Gallus gallus.

In certain example embodiments, the amount of genistein or genistein formulation is effective to (a) modify the intestinal microbiome structure of the avian (e.g., modify to increase the number of probiotic, SCFA producing bacteria, and/or non-pathogenic bacteria (e.g., Bifidobacterium, lactobacillus, L.plantarum, Clostridium bacteria or any combination thereof) and/or decrease the number of pathogenic or non-probiotic bacterium (e.g., E. coli); (b) increase a nutrient and/or mineral bioavailability and/or improve nutrient and/or mineral status in the avian; (c) increase body weight gain of the avian; (d) decrease feed conversion ratio of the avian; (e) support normal avian intestinal health and/or functionality and/or improve intestinal health and/or functionality as optionally measured by intestinal morphology, (including goblet cell number, crypt Paneth cell number), microbiome structure, intestinal health or functionality expression signature (intestinal nutrient transporter expression, intestinal mineral transporter expression, intestinal inflammatory marker expression, intestinal NaK/ATPase exchanger expression, expression of a mineral regulator in the intestine or liver, or any combination thereof), and any combination thereof; (f) correct an iron or Zn deficiency in the avian; (g) modify expression of one or more of the following genes in the avian: DMT1, Ferroportin, DcytB, Bepcidin, Δ-6-desaturase, ZIP6, ZnT7, NF-κβ, TNF-α, AP, SI, NaK/ATPase, and 18S; or (h) any combination thereof.

Described in certain example embodiments herein are formulations comprising an amount of genistein optionally effective, in an avian, to (a) modify the intestinal microbiome structure of the avian (e.g., modify to increase the number of probiotic, SCFA producing bacteria, and/or non-pathogenic bacteria (e.g., Bifidobacterium, lactobacillus, L.plantarum, Clostridium bacteria or any combination thereof) and/or decrease the number of pathogenic or non-probiotic bacterium (e.g., E. coli); (b) increase a nutrient and/or mineral bioavailability and/or improve nutrient and/or mineral status in the avian; (c) increase body weight gain of the avian; (d) decrease feed conversion ratio of the avian; (e) support normal intestinal health and/or functionality and/or improve intestinal health and/or functionality as optionally measured by intestinal morphology, (including goblet cell number, crypt Paneth cell number), microbiome structure, intestinal health or functionality expression signature (intestinal nutrient transporter expression, intestinal mineral transporter expression, intestinal inflammatory marker expression, intestinal NaK/ATPase exchanger expression, expression of a mineral regulator in the intestine or liver, or any combination thereof), and any combination thereof; (f) correct an iron or Zn deficiency in the avian; (g) modify expression of one or more of the following genes in the avian: DMT1, Ferroportin, DcytB, Bepcidin, Δ-6-desaturase, ZIP6, ZnT7, NF-κβ, TNF-α, AP, SI, NaK/ATPase, and 18S; or (h) any combination thereof.

In certain example embodiments, the amount of genistein ranges from about 0.1 wt. % to 5 wt. %, optionally about 0.1 wt. % to 1 wt. %, 1.25 wt. % to about 2.5 wt. %, or about 1 wt. % to 5 wt. % of the formulation or is included in the formulation at an amount such that the amount of genistein in a total diet of a subject to which it is administered ranges from about 0.1 wt. % to 5 wt. %, optionally about 0.1 wt. % to 1 wt. %, 1.25 wt. % to about 2.5 wt. %, or about 1 wt. % to 5 wt. % of the total diet.

In certain example embodiments, the formulation is a feed formulation or dietary supplement.

In certain example embodiments, at least part or all of the genistein is from soybeans included in the formulation. In certain example embodiments, the avian is a neonate, a young non-avian, an adolescent avian, or a mature adult avian.

In certain example embodiments, the subject is an avian of the genus Gallus, Melegris, Dromaius, Struthio, Phoenicopterus, Callipepla, Cyrtonyx, Dactylortyx, Philortyx, Colinus, Odontophorus, Oreortyx, Rhynchortyx, Coturnix, Synoicus, Perdicula, or Ophrysia. In certain example embodiments, the avian is Gallus gallus.

In certain example embodiments, the formulation is adapted for in ovo administration, intraamniotic administration, or oral administration.

Described in certain example embodiments herein are kits comprising a formulation as described in any one of the preceding paragraphs and elsewhere herein.

Described in certain example embodiments herein are uses of the formulations and/or kits to (a) modify the intestinal microbiome structure of the avian (e.g., modify to increase the number of probiotic, SCFA producing bacteria, and/or non-pathogenic bacteria (e.g., Bifidobacterium, lactobacillus, L.plantarum, Clostridium bacteria or any combination thereof) and/or decrease the number of pathogenic or non-probiotic bacterium (e.g., E. coli); (b) increase a nutrient and/or mineral bioavailability and/or improve nutrient and/or mineral status in the avian; (c) increase body weight gain of the avian; (d) decrease feed conversion ratio of the avian; (e) support normal (e.g., non-diseased) avian intestinal health and/or functionality and/or improve intestinal health and/or functionality as optionally measured by intestinal morphology, (including goblet cell number, crypt Paneth cell number), microbiome structure, intestinal health or functionality expression signature (intestinal nutrient transporter expression, intestinal mineral transporter expression, intestinal inflammatory marker expression, intestinal NaK/ATPase exchanger expression, expression of a mineral regulator in the intestine or liver, or any combination thereof), and any combination thereof; (f) correct an iron or Zn deficiency in the avian; (g) modify expression of one or more of the following genes in the avian: DMT1, Ferroportin, DcytB, Bepcidin, Δ-6-desaturase, ZIP6, ZnT7, NF-κβ, TNF-α, AP, SI, NaK/ATPase, and 18S; or (h) any combination thereof.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 - Effect of the intraamniotic administration of experimental solutions on intestinal and liver (hepcidin) gene expression. Values are the means ± SEM, n = 6. ^{a-c} Per gene, treatments groups not indicated by the same letter are significantly different (p < 0.05). DcytB, Duodenal cytochrome b; DMT1, Divalent metal transporter 1; ZIP6, Zinc Transport Protein 6; ZnT7, Zinc transporter 7; AP, Amino peptidase; SI, Sucrose isomaltase; NaK/ATPase, Sodium, Potassium and Adenosine triphosphate; NF_{K}B 1, Nuclear factor Kappa B Subunit 1; TNF-α, Tumor necrosis factor-alpha.

FIG. 2 - Effects of intra-amniotic injections on duodenal genera and species-level bacterial populations (day of hatch). Values are means ± SEM, n = 5. ^{a,b} Per bacterial category, treatments groups that do not share any letters are significantly different (p < 0.05).

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y‘, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y‘, and ‘greater than z’. In addition, the phrase ‘“about ‘x’ to ‘y″’, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F.M. Ausubel et al. eds.); the series Methods in Enzymology (Academic Press, Inc.): PCR 2: A Practical Approach (1995) (M.J. MacPherson, B.D. Hames, and G.R. Taylor eds.): Antibodies, A Laboratory Manual (1988) (Harlow and Lane, eds.): Antibodies A Laboratory Manual, 2^{nd} edition 2013 (E.A. Greenfield ed.); Animal Cell Culture (1987) (R.I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th} Ed. 2021, published by McGraw Hill; Isik et la., Genetic Data Analysis for Plant and Animal Breeding. First ed. 2017. published by Springer International Publishing AG; Green, E. L. Genetics and Probability in Animal Breeding Experiments. 2014, published by Palgrave; Bourdon, R. M. Understanding Animal Breeding. 2000 2^{nd} Ed. published by Prentice Hall; Pal and Chakravarty. Genetics and Breeding for Disease Resistance of Livestock. First Ed. 2019, published by Academic Press; Fasso, D. Classification of Genetic Variance in Animals. First Ed. 2015, published by Callisto Reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5^{th} Ed. 2005, published by Springer-Verlag, New York; Meneely, P. Genetic Analysis: Genes, Genomes, and Networks in Eukaryotes. 3^{rd} Ed. 2020, published by Oxford University Press.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g., given data set, art accepted standard, and/or with e.g., a given confidence interval (e.g., 90%, 95%, or more confidence interval from the mean), such as variations of +/-10% or less, +/-5% or less, +/-1% or less, and +/-0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g., plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably an avian, and more preferably a poultry species. Tissues, cells and their progeny of a biological entity obtained in vivo or cultured in vitro are also encompassed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g., by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra abdominal, intra-amniotic, intraarterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used interchangeably herein, the terms “sufficient” and “effective,” can refer to an amount (e.g., mass, volume, dosage, concentration, and/or time period) needed to achieve one or more desired result(s). For example, a therapeutically effective amount refers to an amount needed to achieve one or more therapeutic effects. Specific desired effects are discussed in greater detail and context herein.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The use of antimicrobials in various livestock industries, including poultry, to prevent and treat disease has been under increasing scrutiny over the past few decades for their role in promoting antimicrobial resistance in key animal and human pathogens. Indeed, over the past several decades poultry producers have faced the implementation of strict government guidelines drastically limiting the use of antimicrobials in production. Several prominent classes of antibiotics, e.g., fluoroquinolones and cephalosporins, important in human medicine have been phased out of use in poultry production. Given the continued pressure to reduce the use of antimicrobials for food production, particularly as growth promoters by preventing sub clinical levels of infection, there exists a need for alternatives to antimicrobials for poultry and livestock production.

With that said, embodiments disclosed herein can provide genistein formulations and methods of using said formulations to improve avian, and more particularly, poultry health. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Formulations

Described in certain example embodiments herein are formulations that contain an amount of genistein optionally effective, in an avian, to modify the intestinal microbiome structure of the avian (e.g., modify to increase the number of probiotic, SCFA producing bacteria, and/or non-pathogenic bacteria (e.g., Bifidobacterium, lactobacillus, L. plantarum, Clostridium bacteria or any combination thereof) and/or decrease the number of pathogenic or non-probiotic bacterium (e.g., E. coli); increase a nutrient and/or mineral bioavailability and/or improve nutrient and/or mineral status in the avian; increase body weight gain of the avian; decrease feed conversion ratio of the avian; support normal intestinal health and/or functionality and/or improve intestinal health and/or functionality as optionally measured by intestinal morphology, (including goblet cell number, crypt Paneth cell number), microbiome structure, intestinal health or functionality expression signature (intestinal nutrient transporter expression, intestinal mineral transporter expression, intestinal inflammatory marker expression, intestinal NaK/ATPase exchanger expression, expression of a mineral regulator in the intestine or liver, or any combination thereof), and any combination thereof; correct an iron or Zn deficiency in the avian; modify expression of one or more of the following genes in the avian: DMT1, Ferroportin, DcytB, Bepcidin, Δ-6-desaturase, ZIP6, ZnT7, NF-κβ, TNF-α, AP, SI, NaK/ATPase, and 18S; or any combination thereof.

In some embodiments, the amount of genistein ranges from about 0.1% to 5% by weight (wt. %), optionally about 0.1 wt. % to 1 wt. %, 1.25 wt. % to about 2.5 wt. %, or 1 wt. % to 5 wt. % of the formulation or is included in the formulation at an amount such that the amount of genistein in a total diet of an avian to which it is administered ranges from about 0.1% to 5% by weight (wt. %), optionally about 0.1 wt. % to 1 wt. %, 1.25 wt. % to about 2.5 wt. %, or 1 wt. % to 5 wt. %.

In some embodiments, the amount of genistein in the formulation is about 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, 0.6 wt. %, 0.61 wt. %, 0.62 wt. %, 0.63 wt. %, 0.64 wt. %, 0.65 wt. %, 0.66 wt. %, 0.67 wt. %, 0.68 wt. %, 0.69 wt. %, 0.7 wt. %, 0.71 wt. %, 0.72 wt. %, 0.73 wt. %, 0.74 wt. %, 0.75 wt. %, 0.76 wt. %, 0.77 wt. %, 0.78 wt. %, 0.79 wt. %, 0.8 wt. %, 0.81 wt. %, 0.82 wt. %, 0.83 wt. %, 0.84 wt. %, 0.85 wt. %, 0.86 wt. %, 0.87 wt. %, 0.88 wt. %, 0.89 wt. %, 0.9 wt. %, 0.91 wt. %, 0.92 wt. %, 0.93 wt. %, 0.94 wt. %, 0.95 wt. %, 0.96 wt. %, 0.97 wt. %, 0.98 wt. %, 0.99 wt. %, 1 wt. %, 1.05 wt. %, 1.1 wt. %, 1.15 wt. %, 1.2 wt. %, 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, 2.5 wt. %, 2.55 wt. %, 2.6 wt. %, 2.65 wt. %, 2.7 wt. %, 2.75 wt. %, 2.8 wt. %, 2.85 wt. %, 2.9 wt. %, 2.95 wt. %, 3 wt. %, 3.05 wt. %, 3.1 wt. %, 3.15 wt. %, 3.2 wt. %, 3.25 wt. %, 3.3 wt. %, 3.35 wt. %, 3.4 wt. %, 3.45 wt. %, 3.5 wt. %, 3.55 wt. %, 3.6 wt. %, 3.65 wt. %, 3.7 wt. %, 3.75 wt. %, 3.8 wt. %, 3.85 wt. %, 3.9 wt. %, 3.95 wt. %, 4 wt. %, 4.05 wt. %, 4.1 wt. %, 4.15 wt. %, 4.2 wt. %, 4.25 wt. %, 4.3 wt. %, 4.35 wt. %, 4.4 wt. %, 4.45 wt. %, 4.5 wt. %, 4.55 wt. %, 4.6 wt. %, 4.65 wt. %, 4.7 wt. %, 4.75 wt. %, 4.8 wt. %, 4.85 wt. %, 4.9 wt. %, 4.95 wt. %, to/or 5 wt. % of the formulation or is otherwise included in the formulation at an amount such that the amount of genistein in a total diet of an avian to which it is administered is about 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, 0.6 wt. %, 0.61 wt. %, 0.62 wt. %, 0.63 wt. %, 0.64 wt. %, 0.65 wt. %, 0.66 wt. %, 0.67 wt. %, 0.68 wt. %, 0.69 wt. %, 0.7 wt. %, 0.71 wt. %, 0.72 wt. %, 0.73 wt. %, 0.74 wt. %, 0.75 wt. %, 0.76 wt. %, 0.77 wt. %, 0.78 wt. %, 0.79 wt. %, 0.8 wt. %, 0.81 wt. %, 0.82 wt. %, 0.83 wt. %, 0.84 wt. %, 0.85 wt. %, 0.86 wt. %, 0.87 wt. %, 0.88 wt. %, 0.89 wt. %, 0.9 wt. %, 0.91 wt. %, 0.92 wt. %, 0.93 wt. %, 0.94 wt. %, 0.95 wt. %, 0.96 wt. %, 0.97 wt. %, 0.98 wt. %, 0.99 wt. %, 1 wt. %, 1.05 wt. %, 1.1 wt. %, 1.15 wt. %, 1.2 wt. %, 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, 2.5 wt. %, 2.55 wt. %, 2.6 wt. %, 2.65 wt. %, 2.7 wt. %, 2.75 wt. %, 2.8 wt. %, 2.85 wt. %, 2.9 wt. %, 2.95 wt. %, 3 wt. %, 3.05 wt. %, 3.1 wt. %, 3.15 wt. %, 3.2 wt. %, 3.25 wt. %, 3.3 wt. %, 3.35 wt. %, 3.4 wt. %, 3.45 wt. %, 3.5 wt. %, 3.55 wt. %, 3.6 wt. %, 3.65 wt. %, 3.7 wt. %, 3.75 wt. %, 3.8 wt. %, 3.85 wt. %, 3.9 wt. %, 3.95 wt. %, 4 wt. %, 4.05 wt. %, 4.1 wt. %, 4.15 wt. %, 4.2 wt. %, 4.25 wt. %, 4.3 wt. %, 4.35 wt. %, 4.4 wt. %, 4.45 wt. %, 4.5 wt. %, 4.55 wt. %, 4.6 wt. %, 4.65 wt. %, 4.7 wt. %, 4.75 wt. %, 4.8 wt. %, 4.85 wt. %, 4.9 wt. %, 4.95 wt. %, to/or 5 wt. %.

In some embodiments, the amount of genistein in the formulation is about 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, 0.6 wt. %, 0.61 wt. %, 0.62 wt. %, 0.63 wt. %, 0.64 wt. %, 0.65 wt. %, 0.66 wt. %, 0.67 wt. %, 0.68 wt. %, 0.69 wt. %, 0.7 wt. %, 0.71 wt. %, 0.72 wt. %, 0.73 wt. %, 0.74 wt. %, 0.75 wt. %, 0.76 wt. %, 0.77 wt. %, 0.78 wt. %, 0.79 wt. %, 0.8 wt. %, 0.81 wt. %, 0.82 wt. %, 0.83 wt. %, 0.84 wt. %, 0.85 wt. %, 0.86 wt. %, 0.87 wt. %, 0.88 wt. %, 0.89 wt. %, 0.9 wt. %, 0.91 wt. %, 0.92 wt. %, 0.93 wt. %, 0.94 wt. %, 0.95 wt. %, 0.96 wt. %, 0.97 wt. %, 0.98 wt. %, 0.99 wt. %, to/or 1% wt. % of the formulation or is otherwise included in the formulation at an amount such that the amount of genistein in a total diet of an avian to which it is administered is about 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, 0.6 wt. %, 0.61 wt. %, 0.62 wt. %, 0.63 wt. %, 0.64 wt. %, 0.65 wt. %, 0.66 wt. %, 0.67 wt. %, 0.68 wt. %, 0.69 wt. %, 0.7 wt. %, 0.71 wt. %, 0.72 wt. %, 0.73 wt. %, 0.74 wt. %, 0.75 wt. %, 0.76 wt. %, 0.77 wt. %, 0.78 wt. %, 0.79 wt. %, 0.8 wt. %, 0.81 wt. %, 0.82 wt. %, 0.83 wt. %, 0.84 wt. %, 0.85 wt. %, 0.86 wt. %, 0.87 wt. %, 0.88 wt. %, 0.89 wt. %, 0.9 wt. %, 0.91 wt. %, 0.92 wt. %, 0.93 wt. %, 0.94 wt. %, 0.95 wt. %, 0.96 wt. %, 0.97 wt. %, 0.98 wt. %, 0.99 wt. %, to/or 1% wt. %.

In some embodiments, the amount of genistein in the formulation is about 1 wt. %, 1.05 wt. %, 1.1 wt. %, 1.15 wt. %, 1.2 wt. %, 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, 2.5 wt. %, 2.55 wt. %, 2.6 wt. %, 2.65 wt. %, 2.7 wt. %, 2.75 wt. %, 2.8 wt. %, 2.85 wt. %, 2.9 wt. %, 2.95 wt. %, 3 wt. %, 3.05 wt. %, 3.1 wt. %, 3.15 wt. %, 3.2 wt. %, 3.25 wt. %, 3.3 wt. %, 3.35 wt. %, 3.4 wt. %, 3.45 wt. %, 3.5 wt. %, 3.55 wt. %, 3.6 wt. %, 3.65 wt. %, 3.7 wt. %, 3.75 wt. %, 3.8 wt. %, 3.85 wt. %, 3.9 wt. %, 3.95 wt. %, 4 wt. %, 4.05 wt. %, 4.1 wt. %, 4.15 wt. %, 4.2 wt. %, 4.25 wt. %, 4.3 wt. %, 4.35 wt. %, 4.4 wt. %, 4.45 wt. %, 4.5 wt. %, 4.55 wt. %, 4.6 wt. %, 4.65 wt. %, 4.7 wt. %, 4.75 wt. %, 4.8 wt. %, 4.85 wt. %, 4.9 wt. %, 4.95 wt. %, to/or 5 wt. % of the formulation or is otherwise included in the formulation at an amount such that the amount of genistein in a total diet of an avian to which it is administered is about 1 wt. %, 1.05 wt. %, 1.1 wt. %, 1.15 wt. %, 1.2 wt. %, 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, 2.5 wt. %, 2.55 wt. %, 2.6 wt. %, 2.65 wt. %, 2.7 wt. %, 2.75 wt. %, 2.8 wt. %, 2.85 wt. %, 2.9 wt. %, 2.95 wt. %, 3 wt. %, 3.05 wt. %, 3.1 wt. %, 3.15 wt. %, 3.2 wt. %, 3.25 wt. %, 3.3 wt. %, 3.35 wt. %, 3.4 wt. %, 3.45 wt. %, 3.5 wt. %, 3.55 wt. %, 3.6 wt. %, 3.65 wt. %, 3.7 wt. %, 3.75 wt. %, 3.8 wt. %, 3.85 wt. %, 3.9 wt. %, 3.95 wt. %, 4 wt. %, 4.05 wt. %, 4.1 wt. %, 4.15 wt. %, 4.2 wt. %, 4.25 wt. %, 4.3 wt. %, 4.35 wt. %, 4.4 wt. %, 4.45 wt. %, 4.5 wt. %, 4.55 wt. %, 4.6 wt. %, 4.65 wt. %, 4.7 wt. %, 4.75 wt. %, 4.8 wt. %, 4.85 wt. %, 4.9 wt. %, 4.95 wt. %, to/or 5 wt. %.

In some embodiments, the amount of genistein in the formulation is about 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, to/or 2.5 wt. % or is otherwise included in the formulation at an amount such that the amount of genistein in a total diet of an avian to which it is administered is about 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, to/or 2.5 wt. %.

In some embodiments, the amount of genistein is present in the formulation such that the amount of genistein in the total diet of an avian to which the formulation is administered to is about 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, 0.6 wt. %, 0.61 wt. %, 0.62 wt. %, 0.63 wt. %, 0.64 wt. %, 0.65 wt. %, 0.66 wt. %, 0.67 wt. %, 0.68 wt. %, 0.69 wt. %, 0.7 wt. %, 0.71 wt. %, 0.72 wt. %, 0.73 wt. %, 0.74 wt. %, 0.75 wt. %, 0.76 wt. %, 0.77 wt. %, 0.78 wt. %, 0.79 wt. %, 0.8 wt. %, 0.81 wt. %, 0.82 wt. %, 0.83 wt. %, 0.84 wt. %, 0.85 wt. %, 0.86 wt. %, 0.87 wt. %, 0.88 wt. %, 0.89 wt. %, 0.9 wt. %, 0.91 wt. %, 0.92 wt. %, 0.93 wt. %, 0.94 wt. %, 0.95 wt. %, 0.96 wt. %, 0.97 wt. %, 0.98 wt. %, 0.99 wt. %, 1 wt. %, 1.05 wt. %, 1.1 wt. %, 1.15 wt. %, 1.2 wt. %, 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, 2.5 wt. %, 2.55 wt. %, 2.6 wt. %, 2.65 wt. %, 2.7 wt. %, 2.75 wt. %, 2.8 wt. %, 2.85 wt. %, 2.9 wt. %, 2.95 wt. %, 3 wt. %, 3.05 wt. %, 3.1 wt. %, 3.15 wt. %, 3.2 wt. %, 3.25 wt. %, 3.3 wt. %, 3.35 wt. %, 3.4 wt. %, 3.45 wt. %, 3.5 wt. %, 3.55 wt. %, 3.6 wt. %, 3.65 wt. %, 3.7 wt. %, 3.75 wt. %, 3.8 wt. %, 3.85 wt. %, 3.9 wt. %, 3.95 wt. %, 4 wt. %, 4.05 wt. %, 4.1 wt. %, 4.15 wt. %, 4.2 wt. %, 4.25 wt. %, 4.3 wt. %, 4.35 wt. %, 4.4 wt. %, 4.45 wt. %, 4.5 wt. %, 4.55 wt. %, 4.6 wt. %, 4.65 wt. %, 4.7 wt. %, 4.75 wt. %, 4.8 wt. %, 4.85 wt. %, 4.9 wt. %, 4.95 wt. %, to/or 5 wt. %.

In some embodiments, the amount of genistein is present in the formulation such that the amount of genistein in the total diet of an avian to which the formulation is administered to is about is about 1 wt. %, 1.05 wt. %, 1.1 wt. %, 1.15 wt. %, 1.2 wt. %, 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, 2.5 wt. %, 2.55 wt. %, 2.6 wt. %, 2.65 wt. %, 2.7 wt. %, 2.75 wt. %, 2.8 wt. %, 2.85 wt. %, 2.9 wt. %, 2.95 wt. %, 3 wt. %, 3.05 wt. %, 3.1 wt. %, 3.15 wt. %, 3.2 wt. %, 3.25 wt. %, 3.3 wt. %, 3.35 wt. %, 3.4 wt. %, 3.45 wt. %, 3.5 wt. %, 3.55 wt. %, 3.6 wt. %, 3.65 wt. %, 3.7 wt. %, 3.75 wt. %, 3.8 wt. %, 3.85 wt. %, 3.9 wt. %, 3.95 wt. %, 4 wt. %, 4.05 wt. %, 4.1 wt. %, 4.15 wt. %, 4.2 wt. %, 4.25 wt. %, 4.3 wt. %, 4.35 wt. %, 4.4 wt. %, 4.45 wt. %, 4.5 wt. %, 4.55 wt. %, 4.6 wt. %, 4.65 wt. %, 4.7 wt. %, 4.75 wt. %, 4.8 wt. %, 4.85 wt. %, 4.9 wt. %, 4.95 wt. %, to/or 5 wt. %.

In some embodiments, the amount of genistein is present in the formulation such that the amount of genistein in the total diet of an avian to which the formulation is administered to is about 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, to/or 2.5 wt. %.

In some embodiments, the amount of genistein in the formulation for in ovo or intra amniotic administration is about 1 wt. %, 1.05 wt. %, 1.1 wt. %, 1.15 wt. %, 1.2 wt. %, 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, 2.5 wt. %, 2.55 wt. %, 2.6 wt. %, 2.65 wt. %, 2.7 wt. %, 2.75 wt. %, 2.8 wt. %, 2.85 wt. %, 2.9 wt. %, 2.95 wt. %, 3 wt. %, 3.05 wt. %, 3.1 wt. %, 3.15 wt. %, 3.2 wt. %, 3.25 wt. %, 3.3 wt. %, 3.35 wt. %, 3.4 wt. %, 3.45 wt. %, 3.5 wt. %, 3.55 wt. %, 3.6 wt. %, 3.65 wt. %, 3.7 wt. %, 3.75 wt. %, 3.8 wt. %, 3.85 wt. %, 3.9 wt. %, 3.95 wt. %, 4 wt. %, 4.05 wt. %, 4.1 wt. %, 4.15 wt. %, 4.2 wt. %, 4.25 wt. %, 4.3 wt. %, 4.35 wt. %, 4.4 wt. %, 4.45 wt. %, 4.5 wt. %, 4.55 wt. %, 4.6 wt. %, 4.65 wt. %, 4.7 wt. %, 4.75 wt. %, 4.8 wt. %, 4.85 wt. %, 4.9 wt. %, 4.95 wt. %, to/or 5 wt. %.

In some embodiments, the amount of genistein in the formulation for in ovo or intra amniotic administration is about 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, to/or 2.5 wt. %.

In some embodiments, the formulation is a feed formulation or feed supplement (dietary supplement). In some embodiments, the formulation is a feed additive.

In some embodiments, formulation includes one or more sources of genistein. In some embodiments, the source of genistein is soybeans, fava beans, lupin, kudzu, psoralea, or any combination thereof. In some embodiments, at least part or all of the genistein is from soybeans included in the formulation.

In some embodiments, the avian is a neonate, a young non-avian, an adolescent avian, or a mature adult avian. In some embodiments, the avian of the genus Gallus, Melegris, Dromaius, Struthio, Phoenicopterus, Callipepla, Cyrtonyx, Dactylortyx, Philortyx, Colinus, Odontophorus, Oreortyx, Rhynchortyx, Coturnix, Synoicus, Perdicula, or Ophrysia. In some embodiments, the avian is Gallus gallus.

In some embodiments, the formulation is a solid, semi solid, or liquid. In some embodiments, formulation is adapted for in ovo administration, intraamniotic administration, or oral administration.

In some embodiments, the feed formulation is formulated for avians. In some embodiments, the formulation is a feed formulation that is formulated as a complete diet. In some embodiments, the formulation is a feed supplement. In some embodiments, the formulation is a feed additive. In some embodiments, the feed formulation, feed additive, or feed supplement is a solid, a semi-solid, or a liquid. In some embodiments, the feed formulation, feed additive, or feed supplement is a powder, pellet, granule, lick block, gel, paste, foam, liquid adapted for aerosolization, and/or the like. As used herein, “complete diet” refers to a feed formulation that contains all the nutrients, calories, minerals, vitamins, and other components needed to meet the dietary requirements of an animal without any additional feed sources and intended to be fed as the entire food source fir the animal. As used herein “feed additive” is an extra nutrient or non-nutrient component that is provided or can be added to a feed or diet that is beyond or in addition to the basic nutritional components of a feed or diet. Feed additives generally fall or can include components that fall into five basic categories: technological additives (e.g., preservatives, antioxidants, emulsifiers, stabilizing agents, acidity regulators, binding agents, silage additives, and/or the like), sensory additives (e.g., flavors, colorants, and/or the like), nutritional additives (e.g., vitamins, minerals, amino acids, trace elements and other minerals, and/or the like), zootechnical additives (e.g., digestibility enhancers, enzymes, intestinal health enhancers, microbes, fiber, and/or the like), and pharmaceuticals. Technological additives are substances or compositions that serves a technological purpose in a feed formulation. Sensory additives are substances or compositions that improve or otherwise changes the organoleptic properties of the feed, or the visual characteristics of the food or other product derived from animals. Nutritional additives are any nutrient substance or component. Zootechnical additives are substances and compositions that improve, favorably affect, or otherwise modify the health and/or performance of an animal and/or modify their impact on the environment. It will be appreciated that any one particular feed additive or component can fall into more than one category. Feed additives can be added to a feed formulation at any stage during feed formulation production (such as being provided in or as a pre-mix) or provided to an animal as a feed supplement that is separate from the feed formulation and added at the point of feeding. As used herein, “feed supplement” refers to a composition formulated for consumption by an animal and intended to be fed undiluted as a supplement to other feeds or offered free choice with other parts of the ration separately available or further diluted and mixed to produce a complete feed ration. A feed additive are intended to be fully incorporated into a feed formulation, while feed supplements indented to be stand-alone compositions that can be fed free choice or mixed in with a feed ration at point of feeding or can be mixed into a feed formulation to make a complete feed formulation. A feed supplement can include one or more feed additive(s). Feed supplement is used interchangeably herein with dietary supplement.

In some embodiments, the feed formulation is formulated to meet the nutritional requirements of a specific age or life-stage or provide some other benefit that is specific to age or life stage. In some embodiments, the feed formulation is formulated to support or meet specific requirements of an animal in a diseased or otherwise non-healthy or normal state. In some embodiments, the feed formulation is formulated to enhance or support the performance of an animal.

In some embodiments, the formulation is effective to modify the intestinal microbiome structure of the avian. As used herein “microbiome structure” refers to the profile of microbiota present in a defined region in or on an organism, such as an organ or tissue. The microbiome structure can include pathogenic and nonpathogenic microbiota. In some embodiments, the formulation is effective to increase the number of probiotic, short-chain fatty acid (SCFA) producing bacterial, and/or non-pathogenic bacteria in the intestinal microbiome. In some embodiments, the formulation is effective to increase the number of probiotic, short-chain fatty acid (SCFA) producing bacterial, and/or non-pathogenic bacteria relative to a control. In some embodiments, the formulation is effective to increase the number of probiotic, short-chain fatty acid (SCFA) producing bacterial, and/or non-pathogenic bacteria relative to the number of pathogenic bacteria in the intestinal microbiome. In some embodiments, the formulation is effective to reduce the number of pathogenic bacteria in the intestinal microbiome. In some embodiments, the formulation is effective to increase the number of probiotic, short-chain fatty acid (SCFA) producing bacterial, and/or non-pathogenic bacteria and reduce the number of pathogenic bacteria in the intestinal microbiome. In some embodiments, the number of different probiotic, short-chain fatty acid (SCFA) producing bacterial, and/or non-pathogenic bacteria species and/or subspecies increases.

Exemplary of probiotic, short-chain fatty acid (SCFA) producing bacterial, and/or non-pathogenic bacteria include, without limitation, bacteria of the genus Bifidobacterium (e.g., B. longum, B. subtilis, B., animalis, B. bifidum), Lactobacillus (e.g., L. reuteri, L. salivarious, L. bifermentans, L. acidophilus, etc.) Streptococcus (e.g., S. faecium), Enterococcus, Enterococcaceae, Campylobacter (C. jejuni and C. coli) Clostridiaceae, Candidatus Arothomitus, Weisella, Ruminococcus, Ruminococcaceae, Eubacterium (e.g., E. Hallii), Bacillus, Staphylococcaceae, Staphylococcus, Turicibacter, Methylobacterium, Bacteriodetes, Flavibacterum, Fusobacteriaum, Ochrobacterium, Alcaligenes, Escherichia, Hafnia, Corymebacterium, Anaerotruncus, Faecalibacterium, Lachnospirceae, megamonas, Rikenellaceae, Bacteriodetes, Alistipes, Odoribacter, Roseburia, any combination thereof, and or the like.

Exemplary pathogenic bacteria include, without limitation, those of the genus Escherichia, (e.g., E. coli) Salmonella, Shigella, Clostridium (e.g., C. difficile, C. perfringens) Aeromonas, and/or the like.

In some embodiments, the formulation is effective to reduce the number of protozoans, particularly pathogenic protozoans (e.g., Eimeria sp.) in the intestine.

In some embodiments, the formulation is effective to increase a nutrient and/or mineral bioavailability and/or improve nutrient and/or mineral status in the avian. In some embodiments, the nutrient or mineral is zinc and/or iron. In some embodiments, the formulation is effective to increase blood hemoglobin levels in the avian.

In some embodiments, the formulation is effective to reduce the intestinal pH. In some embodiments, the formulation is effective to reduce the intestinal pH to between 5 and 7. In some embodiments, the formulation is effective to reduce the pH of the brush border membrane to between 5 and 7.

In some embodiments, the formulation is effective to correct or mitigate an iron and/or Zn deficiency in the avian.

In some embodiments, the formulation is effective to support normal avian intestinal health and/or functionality and/or improve intestinal health and/or functionality as optionally measured by intestinal morphology, (including goblet cell number, crypt Paneth cell number), microbiome structure, intestinal health or functionality expression signature (intestinal nutrient transporter expression, intestinal mineral transporter expression, intestinal inflammatory marker expression, intestinal NaK/ATPase exchanger expression, expression of a mineral regulator in the intestine or liver, or any combination thereof), and any combination thereof.

As used herein a “signature” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any of gene or genes, protein or proteins, or epigenetic element(s) may be substituted. As used herein, the terms “signature”, “expression profile”, or “expression program” may be used interchangeably. It is to be understood that also when referring to proteins (e.g., differentially expressed proteins), such may fall within the definition of “gene” signature. Levels of expression or activity or prevalence may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g. blood samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

It is to be understood that “differentially expressed” genes/proteins include genes/proteins which are up- or down-regulated as well as genes/proteins which are turned on or off. When referring to up-or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

Various aspects and embodiments of the invention may involve analyzing gene signatures, protein signature, and/or other genetic or epigenetic signature based on single cell analyses (e.g., single cell RNA sequencing) or alternatively based on cell population analyses, which are generally known in the art. In one embodiment, the signature genes and/or proteins are detected by PCR based DNA and RNA analysis, sequencing based DNA and RNA analysis immunofluorescence, immunohistochemistry, fluorescence activated cell sorting (FACS), mass cytometry (CyTOF), drop-seq, RNA-seq, single cell qPCR, MERFISH (multiplex (in situ) RNA FISH) and/or by in situ hybridization. Other methods including absorbance assays and colorimetric assays are known in the art and may be used herein.

In some embodiments, the expression signature includes or is, without limitation, ZIP1, ZIP6, ZIP8, ZIP14A/14B, ZIP4, ZIP5, TRPV5, TRPV6, Cav1,3, SGLT1, PepT1, Octyb, DMT1, FAB1, Znt1, Znt7, GLUT2, FPN1, HEPH, FAB2, GLUT5, b^{o+}AT, rBAT, B°AT, ASCT2, EAAT3, y+LAT1, 4F2hc, LAT2, TAT2, PAT1, CTR1, SCL3A1, SCL1A4, SUT2, DTDST, SAT1, DRA, ATOX1, Ferroportin, DcytB, Hepcidin, Δ-6-desaturase, SI, one or more ion or ATPAse exchangers (e.g., NaK/ATPase), NF-ĸβ, TNF-α, and any combination thereof.

In some embodiments, the expression signature includes or is composed only of DMT1, Ferroportin, DcytB, Hepcidin, Δ-6-desaturase, ZIP6, ZnT7, NF-κβ, TNF-α, AP, SI, NaK/ATPase, 18S, or any combination thereof.

In some embodiments, the formula is effective to modify expression of one or more genes and/or gene products from the group of ZIP1, ZIP6, ZIP8, ZIP14A/14B, ZIP4, ZIPS, TRPV5, TRPV6, Cav1,3, SGLT1, PepT1, Octyb, DMT1, FAB1, Znt1, Znt7, GLUT2, FPN1, HEPH, FAB2, GLUT5, b^{o+}AT, rBAT, B^{o}AT, ASCT2, EAAT3, y+LAT1, 4F2hc, LAT2, TAT2, PAT1, CTR1, SCL3A1, SCL1A4, SUT2, DTDST, SAT1, DRA, ATOX1, Ferroportin, DcytB, Hepcidin, Δ-6-desaturase, SI, one or more ion or ATPAse exchangers (e.g., NaK/ATPase), NF-κβ, TNF-α, and any combination thereof.

In some embodiments, the formula is effective to modify expression of one or more genes and/or gene products from the group of DMT1, Ferroportin, DcytB, Hepcidin, Δ-6-desaturase, ZIP6, ZnT7, NF-κβ, TNF-α, AP, SI, NaK/ATPase, 18S, or any combination thereof. In some embodiments, the modification to any one gene or gene product is increase in expression and/or activity. In some embodiments, the modification is a decrease in expression of expression and/or activity.

As used herein “increased expression” or “overexpression” are both used to refer to an increased expression of a gene, such as a gene relating to an antigen processing and/or presentation pathway, or gene product thereof in a sample as compared to the expression of said gene or gene product in a suitable control. The term “increased expression” preferably refers to 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control.

As used herein “decreased expression” “reduced expression” or “underexpression” refers to a reduced or decreased expression of a gene, such as a gene relating to nutrient transporter or inflammation, or a gene product thereof in sample as compared to the expression of said gene or gene product in a suitable control. As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In one embodiment, said control is a sample from a healthy individual or otherwise normal individual. The term “reduced expression” preferably refers to at least a 25% reduction, e.g., at least a 30%, 40%, 50%, 60%, 70%, 75%, 80%, 85%, 90%, 95%, 98% or 99% reduction, relative to such control.

In some embodiments, the increase in expression or activity is an increase of 1 to 100 fold or more, such as 1, to/or 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 fold or more.

In some embodiments, the decrease in expression or activity is a decrease of 1 to 100 fold or more, such as 1, to/or 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100 fold or more.

In some embodiments, the formulation is effective to increase body weight gain as measured between any two time points on an individual or as an average of a population and/or relative to a suitable control. In some embodiments, the increase in body weight gain is 0.001-0.01 percent, 0.01-0.1 percent, 0.1-1 percent, or 1 to 100 percent or more.

In some embodiments, the formulation is effective to decrease feed conversion ratio (FCR) of the avian as measured at any time or time points and/or compared to a suitable control. In some embodiments, FCR is decreased 0.001-0.01 percent,0.01-0.1 percent, 0.1-1 percent, or 1 to 100 percent or more.

Increases and decreases can be determined by comparison to a suitable control. A “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

### Methods

Described in several exemplary embodiments herein are method of modifying an intestinal microbiome structure of an avian including administering, to an avian, an amount of genistein or a genistein formulation, optionally a dietary supplement, feed supplement, or feed formulation, wherein the amount of genistein or genistein formulation is effective to modify the intestinal microbiome structure of the avian (e.g., modify to increase the number of probiotic, SCFA producing bacteria, and/or non-pathogenic bacteria (e.g., Bifidobacterium, lactobacillus, L. plantarum, Clostridium bacteria or any combination thereof) and/or decrease the number of pathogenic or non-probiotic bacterium (e.g., E. coli); increase a nutrient and/or mineral bioavailability and/or improve nutrient and/or mineral status in the avian; increase body weight gain of the avian; decrease feed conversion ratio of the avian; support normal avian intestinal health and/or functionality and/or improve intestinal health and/or functionality as optionally measured by intestinal morphology, (including goblet cell number, crypt Paneth cell number), microbiome structure, intestinal health or functionality expression signature (intestinal nutrient transporter expression, intestinal mineral transporter expression, intestinal inflammatory marker expression, intestinal NaK/ATPase exchanger expression, expression of a mineral regulator in the intestine or liver, or any combination thereof), and any combination thereof; correct an iron or Zn deficiency in the avian; modify expression of one or more of the following genes in the avian: DMT1, Ferroportin, DcytB, Hepcidin, Δ-6-desaturase, ZIP6, ZnT7, NF-ĸβ, TNF-α, AP, SI, NaK/ATPase, and 18S; or any combination thereof.

In some embodiments, the method modifies the intestinal microbiome structure of the avian. As used herein “microbiome structure” refers to the profile of microbiota present in a defined region in or on an organism, such as an organ or tissue. The microbiome structure can include pathogenic and nonpathogenic microbiota. In some embodiments, the method increases the number of probiotic, short-chain fatty acid (SCFA) producing bacterial, and/or non-pathogenic bacteria in the intestinal microbiome. In some embodiments, the method increases e the number of probiotic, short-chain fatty acid (SCFA) producing bacterial, and/or non-pathogenic bacteria relative to a control. In some embodiments, the method increases the number of probiotic, short-chain fatty acid (SCFA) producing bacterial, and/or non-pathogenic bacteria relative to the number of pathogenic bacteria in the intestinal microbiome. In some embodiments, the method reduces the number of pathogenic bacteria in the intestinal microbiome. In some embodiments, the method increases the number of probiotic, short-chain fatty acid (SCFA) producing bacterial, and/or non-pathogenic bacteria and reduces the number of pathogenic bacteria in the intestinal microbiome. In some embodiments, the number of different probiotic, short-chain fatty acid (SCFA) producing bacterial, and/or non-pathogenic bacteria species and/or subspecies is increased by the method.

Exemplary of probiotic, short-chain fatty acid (SCFA) producing bacterial, and/or non-pathogenic bacteria include, without limitation, bacteria of the genus Bifidobacterium (e.g., B. longum, B. subtilis, B., animalis, B. bifidum), Lactobacillus (e.g., L. reuteri, L. salivarious, L. bifermentans, L. acidophilus, etc.) Streptococcus (e.g., S. faecium), Enterococcus, Enterococcaceae, Campylobacter (C. jejuni and C. coli) Clostridiaceae, Candidatus Arothomitus, Weisella, Ruminococcus, Ruminococcaceae, Eubacterium (e.g., E. Hallii), Bacillus, Staphylococcaceae, Staphylococcus, Turicibacter, Methylobacterium, Bacteriodetes, Flavibacterum, Fusobacteriaum, Ochrobacterium, Alcaligenes, Escherichia, Hafnia, Corymebacterium, Anaerotruncus, Faecalibacterium, Lachnospirceae, megamonas, Rikenellaceae, Bacteriodetes, Alistipes, Odoribacter, Roseburia, any combination thereof, and or the like.

Exemplary pathogenic bacteria include, without limitation, those of the genus Escherichia, (e.g., E. coli) Salmonella, Shigella, Clostridium (e.g., C. difficile, C. perfringens) Aeromonas, and/or the like.

In some embodiments, the method reduces the number of protozoans, particularly pathogenic protozoans (e.g., Eimeria sp.) in the intestine.

In some embodiments, the method increases a nutrient and/or mineral bioavailability and/or improve nutrient and/or mineral status in the avian. In some embodiments, the nutrient or mineral is zinc and/or iron. In some embodiments, the method increases blood hemoglobin levels in the avian.

In some embodiments, the method reduces the intestinal pH. In some embodiments, the method reduces the intestinal pH to between 5 and 7. In some embodiments, the method reduces the pH of the brush border membrane to between 5 and 7.

In some embodiments, the formulation is effective to correct or mitigate an iron and/or Zn deficiency in the avian.

In some embodiments, the method supports normal avian intestinal health and/or functionality and/or improve intestinal health and/or functionality as optionally measured by intestinal morphology, (including goblet cell number, crypt Paneth cell number), microbiome structure, intestinal health or functionality expression signature (intestinal nutrient transporter expression, intestinal mineral transporter expression, intestinal inflammatory marker expression, intestinal NaK/ATPase exchanger expression, expression of a mineral regulator in the intestine or liver, or any combination thereof), and any combination thereof.

Exemplary expression signatures are described in greater detail with respect to the formulations.

Described in certain example embodiments herein are methods of treating and/or preventing intestinal abnormality, dysbiosis, malabsorption, malnutrition, infection, and/or disease in an avian, the method comprising administering, to the avian, an amount of genistein or a genistein formulation, optionally a dietary supplement, feed supplement, or feed formulation, wherein the amount of genistein or genistein formulation is effective to preventing intestinal abnormality, dysbiosis, malabsorption, malnutrition, infection, and/or disease in the avian.

In some embodiments the amount of genistein administered ranges from about 0.1 wt. % to 5 wt. %, optionally about 0.1 wt. % to 1 wt. %, 1.25 wt. % to about 2.5 wt. %, or 1 wt. % to 5 wt. % of the genistein formulation, of a dietary supplement, of a feed supplement, of a total diet, or of a feed formulation or is otherwise administered at an amount such that the total amount of genistein in a total diet of the avian ranges from about 0.1 wt. % to 5 wt. %, optionally about 0.1 wt. % to 1 wt. %, 1.25 wt. % to about 2.5 wt. %, or 1 wt. % to 5 wt. %. In some embodiments, the amount of genistein administered is about 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, 0.6 wt. %, 0.61 wt. %, 0.62 wt. %, 0.63 wt. %, 0.64 wt. %, 0.65 wt. %, 0.66 wt. %, 0.67 wt. %, 0.68 wt. %, 0.69 wt. %, 0.7 wt. %, 0.71 wt. %, 0.72 wt. %, 0.73 wt. %, 0.74 wt. %, 0.75 wt. %, 0.76 wt. %, 0.77 wt. %, 0.78 wt. %, 0.79 wt. %, 0.8 wt. %, 0.81 wt. %, 0.82 wt. %, 0.83 wt. %, 0.84 wt. %, 0.85 wt. %, 0.86 wt. %, 0.87 wt. %, 0.88 wt. %, 0.89 wt. %, 0.9 wt. %, 0.91 wt. %, 0.92 wt. %, 0.93 wt. %, 0.94 wt. %, 0.95 wt. %, 0.96 wt. %, 0.97 wt. %, 0.98 wt. %, 0.99 wt. %, 1 wt. %, 1.05 wt. %, 1.1 wt. %, 1.15 wt. %, 1.2 wt. %, 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, 2.5 wt. %, 2.55 wt. %, 2.6 wt. %, 2.65 wt. %, 2.7 wt. %, 2.75 wt. %, 2.8 wt. %, 2.85 wt. %, 2.9 wt. %, 2.95 wt. %, 3 wt. %, 3.05 wt. %, 3.1 wt. %, 3.15 wt. %, 3.2 t. %, 3.25 wt. %, 3.3 wt. %, 3.35 wt. %, 3.4 wt. %, 3.45 wt. %, 3.5 wt. %, 3.55 wt. %, 3.6 wt. %, 3.65 wt. %, 3.7 wt. %, 3.75 wt. %, 3.8 wt. %, 3.85 wt. %, 3.9 wt. %, 3.95 wt. %, 4 wt. %, 4.05 wt. %, 4.1 wt. %, 4.15 wt. %, 4.2 wt. %, 4.25 wt. %, 4.3 wt. %, 4.35 wt. %, 4.4 wt. %, 4.45 wt. %, 4.5 wt. %, 4.55 wt. %, 4.6 wt. %, 4.65 wt. %, 4.7 wt. %, 4.75 wt. %, 4.8 wt. %, 4.85 wt. %, 4.9 wt. %, 4.95 wt. %, to/or 5 wt. % of the genistein formulation, of a dietary supplement, of a feed supplement, of a total diet, or of a feed formulation or is otherwise administered at an amount such that the total amount of genistein in a total diet of the avian is about 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, 0.6 wt. %, 0.61 wt. %, 0.62 wt. %, 0.63 wt. %, 0.64 wt. %, 0.65 wt. %, 0.66 wt. %, 0.67 wt. %, 0.68 wt. %, 0.69 wt. %, 0.7 wt. %, 0.71 wt. %, 0.72 wt. %, 0.73 wt. %, 0.74 wt. %, 0.75 wt. %, 0.76 wt. %, 0.77 wt. %, 0.78 wt. %, 0.79 wt. %, 0.8 wt. %, 0.81 wt. %, 0.82 wt. %, 0.83 wt. %, 0.84 wt. %, 0.85 wt. %, 0.86 wt. %, 0.87 wt. %, 0.88 wt. %, 0.89 wt. %, 0.9 wt. %, 0.91 wt. %, 0.92 wt. %, 0.93 wt. %, 0.94 wt. %, 0.95 wt. %, 0.96 wt. %, 0.97 wt. %, 0.98 wt. %, 0.99 wt. %, 1 wt. %, 1.05 wt. %, 1.1 wt. %, 1.15 wt. %, 1.2 wt. %, 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, 2.5 wt. %, 2.55 wt. %, 2.6 wt. %, 2.65 wt. %, 2.7 wt. %, 2.75 wt. %, 2.8 wt. %, 2.85 wt. %, 2.9 wt. %, 2.95 wt. %, 3 wt. %, 3.05 wt. %, 3.1 wt. %, 3.15 wt. %, 3.2 t. %, 3.25 wt. %, 3.3 wt. %, 3.35 wt. %, 3.4 wt. %, 3.45 wt. %, 3.5 wt. %, 3.55 wt. %, 3.6 wt. %, 3.65 wt. %, 3.7 wt. %, 3.75 wt. %, 3.8 wt. %, 3.85 wt. %, 3.9 wt. %, 3.95 wt. %, 4 wt. %, 4.05 wt. %, 4.1 wt. %, 4.15 wt. %, 4.2 wt. %, 4.25 wt. %, 4.3 wt. %, 4.35 wt. %, 4.4 wt. %, 4.45 wt. %, 4.5 wt. %, 4.55 wt. %, 4.6 wt. %, 4.65 wt. %, 4.7 wt. %, 4.75 wt. %, 4.8 wt. %, 4.85 wt. %, 4.9 wt. %, 4.95 wt. %, to/or 5 wt. %.

In some embodiments, the amount of genistein administered is about 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, 0.6 wt. %, 0.61 wt. %, 0.62 wt. %, 0.63 wt. %, 0.64 wt. %, 0.65 wt. %, 0.66 wt. %, 0.67 wt. %, 0.68 wt. %, 0.69 wt. %, 0.7 wt. %, 0.71 wt. %, 0.72 wt. %, 0.73 wt. %, 0.74 wt. %, 0.75 wt. %, 0.76 wt. %, 0.77 wt. %, 0.78 wt. %, 0.79 wt. %, 0.8 wt. %, 0.81 wt. %, 0.82 wt. %, 0.83 wt. %, 0.84 wt. %, 0.85 wt. %, 0.86 wt. %, 0.87 wt. %, 0.88 wt. %, 0.89 wt. %, 0.9 wt. %, 0.91 wt. %, 0.92 wt. %, 0.93 wt. %, 0.94 wt. %, 0.95 wt. %, 0.96 wt. %, 0.97 wt. %, 0.98 wt. %, 0.99 wt. %, to/or 1% of the genistein formulation, of a dietary supplement, of a feed supplement, of a total diet, or of a feed formulation or is otherwise administered at an amount such that the total amount of genistein in a total diet of the avian is about 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, 0.6 wt. %, 0.61 wt. %, 0.62 wt. %, 0.63 wt. %, 0.64 wt. %, 0.65 wt. %, 0.66 wt. %, 0.67 wt. %, 0.68 wt. %, 0.69 wt. %, 0.7 wt. %, 0.71 wt. %, 0.72 wt. %, 0.73 wt. %, 0.74 wt. %, 0.75 wt. %, 0.76 wt. %, 0.77 wt. %, 0.78 wt. %, 0.79 wt. %, 0.8 wt. %, 0.81 wt. %, 0.82 wt. %, 0.83 wt. %, 0.84 wt. %, 0.85 wt. %, 0.86 wt. %, 0.87 wt. %, 0.88 wt. %, 0.89 wt. %, 0.9 wt. %, 0.91 wt. %, 0.92 wt. %, 0.93 wt. %, 0.94 wt. %, 0.95 wt. %, 0.96 wt. %, 0.97 wt. %, 0.98 wt. %, 0.99 wt. %, to/or 1%.

In some embodiments, the amount of genistein administered is about 1 wt. %, 1.05 wt. %, 1.1 wt. %, 1.15 wt. %, 1.2 wt. %, 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, 2.5 wt. %, 2.55 wt. %, 2.6 wt. %, 2.65 wt. %, 2.7 wt. %, 2.75 wt. %, 2.8 wt. %, 2.85 wt. %, 2.9 wt. %, 2.95 wt. %, 3 wt. %, 3.05 wt. %, 3.1 wt. %, 3.15 wt. %, 3.2 wt. %, 3.25 wt. %, 3.3 wt. %, 3.35 wt. %, 3.4 wt. %, 3.45 wt. %, 3.5 wt. %, 3.55 wt. %, 3.6 wt. %, 3.65 wt. %, 3.7 wt. %, 3.75 wt. %, 3.8 wt. %, 3.85 wt. %, 3.9 wt. %, 3.95 wt. %, 4 wt. %, 4.05 wt. %, 4.1 wt. %, 4.15 wt. %, 4.2 wt. %, 4.25 wt. %, 4.3 wt. %, 4.35 wt. %, 4.4 wt. %, 4.45 wt. %, 4.5 wt. %, 4.55 wt. %, 4.6 wt. %, 4.65 wt. %, 4.7 wt. %, 4.75 wt. %, 4.8 wt. %, 4.85 wt. %, 4.9 wt. %, 4.95 wt. %, to/or 5 wt. % of the genistein formulation, of a dietary supplement, of a feed supplement, of a total diet, or of a feed formulation or is otherwise administered at an amount such that the total amount of genistein in a total diet of the avian is about 1 wt. %, 1.05 wt. %, 1.1 wt. %, 1.15 wt. %, 1.2 wt. %, 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, 2.5 wt. %, 2.55 wt. %, 2.6 wt. %, 2.65 wt. %, 2.7 wt. %, 2.75 wt. %, 2.8 wt. %, 2.85 wt. %, 2.9 wt. %, 2.95 wt. %, 3 wt. %, 3.05 wt. %, 3.1 wt. %, 3.15 wt. %, 3.2 wt. %, 3.25 wt. %, 3.3 wt. %, 3.35 wt. %, 3.4 wt. %, 3.45 wt. %, 3.5 wt. %, 3.55 wt. %, 3.6 wt. %, 3.65 wt. %, 3.7 wt. %, 3.75 wt. %, 3.8 wt. %, 3.85 wt. %, 3.9 wt. %, 3.95 wt. %, 4 wt. %, 4.05 wt. %, 4.1 wt. %, 4.15 wt. %, 4.2 wt. %, 4.25 wt. %, 4.3 wt. %, 4.35 wt. %, 4.4 wt. %, 4.45 wt. %, 4.5 wt. %, 4.55 wt. %, 4.6 wt. %, 4.65 wt. %, 4.7 wt. %, 4.75 wt. %, 4.8 wt. %, 4.85 wt. %, 4.9 wt. %, 4.95 wt. %, to/or 5 wt. %.

In some embodiments, the amount of genistein is about 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, to/or 2.5 wt. % of the genistein formulation, of a dietary supplement, of a feed supplement, of a total diet, or of a feed formulation or is otherwise administered at an amount such that the total amount of genistein in a total diet of the avian is about 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, to/or 2.5 wt. %.

In some embodiments the amount of genistein administered is such that the total amount of genistein administered to the avian, such as in a total diet, is about 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, 0.6 wt. %, 0.61 wt. %, 0.62 wt. %, 0.63 wt. %, 0.64 wt. %, 0.65 wt. %, 0.66 wt. %, 0.67 wt. %, 0.68 wt. %, 0.69 wt. %, 0.7 wt. %, 0.71 wt. %, 0.72 wt. %, 0.73 wt. %, 0.74 wt. %, 0.75 wt. %, 0.76 wt. %, 0.77 wt. %, 0.78 wt. %, 0.79 wt. %, 0.8 wt. %, 0.81 wt. %, 0.82 wt. %, 0.83 wt. %, 0.84 wt. %, 0.85 wt. %, 0.86 wt. %, 0.87 wt. %, 0.88 wt. %, 0.89 wt. %, 0.9 wt. %, 0.91 wt. %, 0.92 wt. %, 0.93 wt. %, 0.94 wt. %, 0.95 wt. %, 0.96 wt. %, 0.97 wt. %, 0.98 wt. %, 0.99 wt. %, 1 wt. %, 1.05 wt. %, 1.1 wt. %, 1.15 wt. %, 1.2 wt. %, 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, 2.5 wt. %, 2.55 wt. %, 2.6 wt. %, 2.65 wt. %, 2.7 wt. %, 2.75 wt. %, 2.8 wt. %, 2.85 wt. %, 2.9 wt. %, 2.95 wt. %, 3 wt. %, 3.05 wt. %, 3.1 wt. %, 3.15 wt. %, 3.2 wt. %, 3.25 wt. %, 3.3 wt. %, 3.35 wt. %, 3.4 wt. %, 3.45 wt. %, 3.5 wt. %, 3.55 wt. %, 3.6 wt. %, 3.65 wt. %, 3.7 wt. %, 3.75 wt. %, 3.8 wt. %, 3.85 wt. %, 3.9 wt. %, 3.95 wt. %, 4 wt. %, 4.05 wt. %, 4.1 wt. %, 4.15 wt. %, 4.2 wt. %, 4.25 wt. %, 4.3 wt. %, 4.35 wt. %, 4.4 wt. %, 4.45 wt. %, 4.5 wt. %, 4.55 wt. %, 4.6 wt. %, 4.65 wt. %, 4.7 wt. %, 4.75 wt. %, 4.8 wt. %, 4.85 wt. %, 4.9 wt. %, 4.95 wt. %, to/or 5 wt. %.

In some embodiments the amount of genistein administered is such that the total amount of genistein administered to the avian, such as in a total diet, is about 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, 0.6 wt. %, 0.61 wt. %, 0.62 wt. %, 0.63 wt. %, 0.64 wt. %, 0.65 wt. %, 0.66 wt. %, 0.67 wt. %, 0.68 wt. %, 0.69 wt. %, 0.7 wt. %, 0.71 wt. %, 0.72 wt. %, 0.73 wt. %, 0.74 wt. %, 0.75 wt. %, 0.76 wt. %, 0.77 wt. %, 0.78 wt. %, 0.79 wt. %, 0.8 wt. %, 0.81 wt. %, 0.82 wt. %, 0.83 wt. %, 0.84 wt. %, 0.85 wt. %, 0.86 wt. %, 0.87 wt. %, 0.88 wt. %, 0.89 wt. %, 0.9 wt. %, 0.91 wt. %, 0.92 wt. %, 0.93 wt. %, 0.94 wt. %, 0.95 wt. %, 0.96 wt. %, 0.97 wt. %, 0.98 wt. %, 0.99 wt. %, to/or 1%.

In some embodiments the amount of genistein administered is such that the total amount of genistein administered to the avian, such as in a total diet, is about 1 wt. %, 1.05 wt. %, 1.1 wt. %, 1.15 wt. %, 1.2 wt. %, 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, 2.5 wt. %, 2.55 wt. %, 2.6 wt. %, 2.65 wt. %, 2.7 wt. %, 2.75 wt. %, 2.8 wt. %, 2.85 wt. %, 2.9 wt. %, 2.95 wt. %, 3 wt. %, 3.05 wt. %, 3.1 wt. %, 3.15 wt. %, 3.2 wt. %, 3.25 wt. %, 3.3 wt. %, 3.35 wt. %, 3.4 wt. %, 3.45 wt. %, 3.5 wt. %, 3.55 wt. %, 3.6 wt. %, 3.65 wt. %, 3.7 wt. %, 3.75 wt. %, 3.8 wt. %, 3.85 wt. %, 3.9 wt. %, 3.95 wt. %, 4 wt. %, 4.05 wt. %, 4.1 wt. %, 4.15 wt. %, 4.2 wt. %, 4.25 wt. %, 4.3 wt. %, 4.35 wt. %, 4.4 wt. %, 4.45 wt. %, 4.5 wt. %, 4.55 wt. %, 4.6 wt. %, 4.65 wt. %, 4.7 wt. %, 4.75 wt. %, 4.8 wt. %, 4.85 wt. %, 4.9 wt. %, 4.95 wt. %, to/or 5 wt. %.

In some embodiments the amount of genistein administered is such that the total amount of genistein administered to the avian, such as in a total diet, is about 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, to/or 2.5 wt. %.

In some embodiments, the amount of genistein administered for in ovo or intra amniotic administration is about 1 wt. %, 1.05 wt. %, 1.1 wt. %, 1.15 wt. %, 1.2 wt. %, 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, 2.5 wt. %, 2.55 wt. %, 2.6 wt. %, 2.65 wt. %, 2.7 wt. %, 2.75 wt. %, 2.8 wt. %, 2.85 wt. %, 2.9 wt. %, 2.95 wt. %, 3 wt. %, 3.05 wt. %, 3.1 wt. %, 3.15 wt. %, 3.2 wt. %, 3.25 wt. %, 3.3 wt. %, 3.35 wt. %, 3.4 wt. %, 3.45 wt. %, 3.5 wt. %, 3.55 wt. %, 3.6 wt. %, 3.65 wt. %, 3.7 wt. %, 3.75 wt. %, 3.8 wt. %, 3.85 wt. %, 3.9 wt. %, 3.95 wt. %, 4 wt. %, 4.05 wt. %, 4.1 wt. %, 4.15 wt. %, 4.2 wt. %, 4.25 wt. %, 4.3 wt. %, 4.35 wt. %, 4.4 wt. %, 4.45 wt. %, 4.5 wt. %, 4.55 wt. %, 4.6 wt. %, 4.65 wt. %, 4.7 wt. %, 4.75 wt. %, 4.8 wt. %, 4.85 wt. %, 4.9 wt. %, 4.95 wt. %, to/or 5 wt. %.

In some embodiments, the amount of genistein administered for in ovo or intra amniotic administration is about 1.25 wt. %, 1.3 wt. %, 1.35 wt. %, 1.4 wt. %, 1.45 wt. %, 1.5 wt. %, 1.55 wt. %, 1.6 wt. %, 1.65 wt. %, 1.7 wt. %, 1.75 wt. %, 1.8 wt. %, 1.85 wt. %, 1.9 wt. %, 1.95 wt. %, 2 wt. %, 2.05 wt. %, 2.1 wt. %, 2.15 wt. %, 2.2 wt. %, 2.25 wt. %, 2.3 wt. %, 2.35 wt. %, 2.4 wt. %, 2.45 wt. %, to/or 2.5 wt. %.

In some embodiments the genistein formulation is as described elsewhere herein (see e.g., “Formulations” and Working Examples herein).

In some embodiments the genistein or genistein formulation is administered daily, weekly, monthly, or yearly. In some embodiments, the genistein or genistein formulation is administered every other day, every third day, every fourth day, every fifth day, every sixth day or every seventh day. In some embodiments, the genistein or genistein formulation is administered two, three, four, or more times a day.

The genistein or genistein formulation can be administered to an avian at any age or during embryonic development prior to and/or during hatching.

In some embodiments, the avian is a developing embryo. In some embodiments, the avian is a neonate, a young avian, an adolescent avian, or a mature adult avian. In some embodiments, administration is during embryonic development, optionally directly to a developing embryo or indirectly to a sexually mature avian. In some embodiments, administration is via in ovo injection or intraamniotic injection.

In some embodiments, administration is oral. In some embodiments, administration is internasal (such as by aerosolized spray or mist).

In some embodiments, the avian, is an avian of the genus Gallus, Melegris, Dromaius, Struthio, Phoenicopterus, Callipepla, Cyrtonyx, Dactylortyx, Philortyx, Colinus, Odontophorus, Oreortyx, Rhynchortyx, Coturnix, Synoicus, Perdicula, or Ophrysia. In some embodiments, the avian is Gallus gallus.

In some embodiments, the amount of amount of genistein or genistein formulation is effective to modify the intestinal microbiome structure of the avian (e.g., modify to increase the number of probiotic, SCFA producing bacteria, and/or non-pathogenic bacteria (e.g., Bifidobacterium, lactobacillus, L. plantarum, Clostridium bacteria or any combination thereof) and/or decrease the number of pathogenic or non-probiotic bacterium (e.g., E. coli); increase a nutrient and/or mineral bioavailability and/or improve nutrient and/or mineral status in the avian; increase body weight gain of the avian; decrease feed conversion ratio of the avian; support normal avian intestinal health and/or functionality and/or improve intestinal health and/or functionality as optionally measured by intestinal morphology, (including goblet cell number, crypt Paneth cell number), microbiome structure, intestinal health or functionality expression signature (intestinal nutrient transporter expression, intestinal mineral transporter expression, intestinal inflammatory marker expression, intestinal NaK/ATPase exchanger expression, expression of a mineral regulator in the intestine or liver, or any combination thereof), and any combination thereof; correct an iron or Zn deficiency in the avian; modify expression of one or more of the following genes in the avian: DMT1, Ferroportin, DcytB, Bepcidin, Δ-6-desaturase, ZIP6, ZnT7, NF-κβ, TNF-α, AP, SI, NaK/ATPase, and 18S; or any combination thereof. Other effects and expression signatures and modification thereof by the genistein formulations are described elsewhere herein.

### Hits

Any of the compounds, compositions and formulations described herein or a combination thereof can be presented as a combination kit. As used herein, the terms “combination kit” or “kit of parts” refers to the compounds, compositions and formulations and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions and formulations described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions and formulations described herein or a combination thereof contained therein, safety information regarding the content of the compounds, compositions and formulations described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions and formulations described herein or a combination thereof to a subject in need thereof, such as an avian. As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in °C, and pressure is at or near atmospheric. Standard temperature and pressure are defined as 20° C. and 1 atmosphere.

### Example 1

Genistein is a polyphenolic isoflavone naturally present in a number of staple crops, including soybeans and chickpeas. Many studies have reported genistein to possess a variety of beneficial and protective physiological properties, with effects observed in in vivo models of metabolic syndrome, diabetes, and breast and prostate cancers [1,2]. Biological effects of isoflavone consumption have been attributed to structural similarity and function with human and animal estrogens. Specifically, due to structural similarity to 17b-estradiol, genistein has been observed to possess weak estrogenic activity and exhibit preferential binding to estrogen receptor β [2,3].

The characterization of genistein metabolism and absorption is still ongoing, despite the well-studied physiological effects of genistein and other isoflavones. Dietary isoflavones exist as isoflavone-glycosides and are transformed by intestinal microbiota via bacterial enzymatic action to more potent metabolites, such as equol and O-desmethylangolensin [4]. Thus, individual differences in gut microbiota will consequently be expected to influence the potential for physiological effects associated with isoflavone injestion [5]. Current research has shown genistein administration in mice fed a high fat diet had increased gut bacterial populations associated with decreasing levels of lipopolysaccharide, a potent inflammatory agent [1]. Another recent study has shown that in vitro isoflavones administration promoted short chain fatty acid (SCFA) production due to growth stimulation of SCFA-producing bacteria species, Roseburia and E. hallii (both from Clostridium cluster XIVa) [4]. Additionally, maternal genistein intake perinatally and throughout pregnancy in mice mitigated the harmful effects of a high-fat fed diet in dams and offspring and was associated with an increase in butyrate producing gut bacteria [6]. Increased SCFA production has been associated with inhibition of harmful pathogen growth, decreased intestinal pH, and upregulated brush border membrane (BBM) gene expression [7,8]. Taken together, these effects enhance micronutrient bioavailability and absorption.

Emerging evidence suggests that genistein exposure could be implicated in altered expression of proteins involved in iron (Fe) transport. Genistein significantly increased Fe export through estrogen receptor β-dependent p38 MAPK up-regulation through ceruloplasmin and ferroportin-1 in glial cells [9]. However, another study found genistein treatment of human hepatocytes increased both hepcidin transcription levels and promoter activity (hepcidin decreases intestinal Fe absorption by inhibiting ferroportin) [10].

Despite the investigation of specific health benefits attributed to dietary genistein administration, and subsequent knowledge of genistein ingestion on gut microbiota modulation and Fe transport, there is a paucity of knowledge with regards to how genistein affects the functionality of the small intestine, specifically the brush border member (BBM). The functional capacity (e.g., digestive enzyme production) of the BBM dictates the extent of food hydrolysis and micronutrient uptake. It remains a priority to investigate the interactions between bioactive extracts in the diet and the BBM.

In this Example, the effects of genistein intraamniotic administration on brush border membrane (BBM) functionality, intestinal morphology, intestinal microbiome, and mineral status were studied in vivo in the Gallus gallus. Due to similarities in intestinal morphology, microbiota, and gene homology of duodenal mineral transporters between humans and Gallus gallus, Gallus gallus have been used as a novel animal system to elucidate the physiological effects of plant bioactives and nutritional solutions relevant to human nutrition [11-15]. It was previously demonstrated that daidzein, a major isoflavone found in soybeans with estrogenic effects, affected the expression of BBM Fe-related proteins and intestinal bacterial populations [16,17]. Therefore, the first objective of this study was to assess the effects of genistein on mineral status. To accomplish this objective, Applicant assessed hemoglobin (Hb), expression of duodenal cytochrome B (DcytB, a Fe-specific cytochrome reductase on the luminal side of the enterocyte) and divalent metal transporter 1 (DMT1, primary transporter of Fe²⁺ from the luminal side of the enterocyte), ferroportin (a basolateral exporter of dietary Fe²⁺), liver hepcidin (decreases intestinal Fe absorption by inhibiting ferroportin), as well as ZnT-7 (zinc transporter protein-7) and ZIP6 (zinc transporter) [10]. BBM functionality was evaluated by assessing duodenum morphology and expression of biomarkers of BBM digestive and absorptive ability (AP- aminopeptidase, SI-sucrase isomaltase, and NaK/ATPase). In addition, systemic inflammatory status was evaluated using expression of immunoregulatory cytokines (TNF-a, Tumor necrosis factor-alpha; and NF_{ĸ}B1, Nuclear factor Kappa B Subunit 1). The second objective was to utilize PCR quantification to analyze duodenal microbial populations and 16S rRNA gene sequencing to analyze cecal contents to elucidate potential alterations in intestinal microbiota structure and function from genistein administration. We hypothesize that intraamniotic administration of genistein will improve mineral status, cause favorable alterations in BBM functionality and development, and positively modulate the gut microbiota.

### 2. Materials and Methods

### 2.1. Animals and Study Design

Cornish-cross fertile broiler eggs (Gallus gallus, n = 39) were obtained from a commercial hatchery (Moyer’s chicks, Quakertown, PA, USA). The eggs were incubated under optimal conditions at the Cornell University Animal Science Poultry Farm Incubator [18]. All animal protocols were approved by Cornell University Institutional Animal Care and Use committee (IACUC #2020-0077). At day 17 of embryonic incubation, eggs containing viable embryos were weighed and divided into 5 groups, with all treatment groups assigned eggs of similar weight frequency distribution. Each group was then injected with the specific solution (1 mL per egg) with a 21-gauge needle into the amniotic fluid. Treatments in powder form were separately diluted in DI H₂O. Two treatment groups (1.25, 2.5 wt. %), two controls (H₂O and non-injected), and a positive control (5 wt. % inulin) were administered. After injection, the injection holes were sealed with cellophane tape and the eggs were placed in hatching baskets such that each treatment was equally represented at each incubator location. Immediately upon hatch (day 21), blood was taken for hemoglobin determination and all chicks were then euthanized by CO₂ exposure. The small intestine, pectoral muscle, cecum, and liver were collected, immediately frozen in liquid nitrogen, and then stored in a -80° C. freezer until analysis.

### 2.2. Blood Analysis and Hemoglobin Measurements

Blood was collected using micro-hematocrit heparinized capillary tubes (Fisher Scientific, Waltham, MA, USA). Blood Hb concentrations were determined spectrophotometrically using the QuantiChromTM Hemoglobin Assay (DIHB-250, BioAssay Systems, Hayward, CA, USA) following the kit manufacturer’s instructions.

### 2.3. Isolation of Total RNA From Duodenum and Liver Tissue Samples.

Total RNA was extracted from 30 mg of the proximal duodenal (n=6) or liver tissues (n=6) using Qiagen RNeasy Mini Kit (Qiagen Inc., Valencia, CA) according to the manufacturer’s protocol. All steps were carried out under RNase free conditions. RNA was quantified by absorbance at 260-280 nm using a NanoDrop 2000 (ThermoFisher Scientific, Waltham, MA). RNA was stored at -80° C. until use.

### 2.4. Real-Time Polymerase Chain Reaction (RT-PCR)

As previously described [19]. cDNA was generated using a 20 uL reverse transcriptase (RT) reaction in a BioRad C1000 touch thermocycler using the Improm-II Reverse Transcriptase Kit (Catalog #A1250; Promega, Madison, WI, USA). The reverse transcriptase reaction consisted of 1 µL total RNA template, 10 µM random hexamer primers, and 2 mM of oligo-dT primers. All reactions were performed under the following conditions: 94° C. for 5 minutes, 60 minutes at 42° C., 70° C. for 15 minutes, and hold at 4° C. The concentration of cDNA obtained was determined with a NanoDrop 2000 (ThermoFisher Scientific, Waltham, MA) by measuring the absorbance at 260-280 nm using an extinction coefficient of 33 (for single stranded DNA). Genomic DNA contamination was assessed using a RT-PCR assay for the reference of genes samples.

### 2.4.1 Primer Design

Primer design was conducted as previously described [19]. The primers used in the real-time polymerase chain reactions (RT-PCR) were designed using Real-Time Primer Design Tool software (IDT DNA, Coralvilla, IA) based on 13 gene sequences from GenBank database. The sequences and the description of the primers used in this study are summarized in Table 1.

TABLE 1

The DNA sequences of primers used in this study

| Analyte | Forward Primer (5′- 3′) | Reverse Primer (5′- 3′) | Base Pair | GI Identifier |
|---|---|---|---|---|
| Iron Metabolism | | | | |
| DMT1 | TTGATTCAGAGCCTCCCAT TAG (SEQ ID NO: 1) | GCGAGGAGTAGGCTTGTA TTT (SEQ ID NO: 2) | 101 | 206597489 |
| Ferroportin | CTCAGCAATCACTGGCATCA (SEQ ID NO: 3) | ACTGGGCAACTCCAGAAATAAG (SEQ ID NO: 4) | 98 | 61098365 |
| DcytB | CATGTGCATTCTCTTCCAAAGTC (SEQ ID NO: 5) | CTCCTTGGTGACCGCATTAT (SEQ ID NO: 6) | 103 | 20380692 |
| Hepcidin | AGACGACAATGCAGACTA ACC (SEQ ID NO: 7) | CTGCAGCAATCCCACATTT C (SEQ ID NO: 8) | 132 | SAMN080564 90 |
| Zinc Metabolism | | | | |
| Δ-6-desaturase | GGCGAAAGTCAGCCTATTGA (SEQ ID NO: 9) | AGGTGGGAAGATGAGGAAGA (SEQ ID NO: 10) | 93 | 261865208 |
| ZIP6 | GCTACTGGGTAATGGTGAAGAA (SEQ ID NO: 11) | GCTGTGCCAGAACTGTAGAA (SEQ ID NO: 12) | 380 | 66735072 |
| ZnT7 | GGAAGATGTCAGGATGGTTCA (SEQ ID NO: 13) | CGAAGGACAAATTGAGGCAAAG (SEQ ID NO: 14) | 87 | 56555152 |
| Inflammatory Response | | | | |
| NF-κβ | CACAGCTGGAGGGAAGTA AAT (SEQ ID NO: 15) | TTGAGTAAGGAAGTGAGG TTGAG (SEQ ID NO: 16) | 100 | 2130627 |
| TNF-α | GACAGCCTATGCCAACAAG TA (SEQ ID NO: 17) | TTACAGGAAGGGCAACTC ATC (SEQ ID NO: 18) | 109 | 53854909 |
| BBM Functionality | | | | |
| AP | CGTCAGCCAGTTTGACTAT GTA (SEQ ID NO: 19) | CTCTCAAAGAAGCTGAGG ATGG (SEQ ID NO: 20) | 138 | 45382360 |
| SI | CCAGCAATGCCAGCATATT G (SEQ ID NO: 21) | CGGTTTCTCCTTACCACTT CTT (SEQ ID NO: 22) | 95 | 2246388 |
| NaK/ATPa se | CCTTGGAGGTTTCTTCACCT ATT (SEQ ID NO: 23) | GGTCATCCCACTGAAGTCT AATC (SEQ ID NO: 24) | 92 | 14330321 |
| 18S | GCAAGACGAACTAAAGCG AAAG (SEQ ID NO: 25) | TCGGAACTACGACGGTAT CT (SEQ ID NO: 26) | 100 | 7262899 |

DMT1, Divalent metal transporter 1; DcytB, Duodenal cytochrome b; ZnT7, Zinc transporter 7; ZIP6, Zinc Transport Protein 6; TNF-α, Tumor necrosis factor-alpha; NF_{ĸ}B1, Nuclear factor Kappa B Subunit 1; AP, Amino peptidase; SI, Sucrose isomaltase; NaK/ATPase, Sodium, Potassium and Adenosine triphosphate.

### 2.4.2 Real-Time qPCR Design

Procedures were carried out as previously described [19]. Briefly, RT-qPCR was performed with a Bio-RadCFX96 Touch (Hercules, CA, USA). The 10 µL RT-qPCR mixtures consisted of cDNA (2 µL), 2X BioRad SSO Advanced Universal SYBR Green Supermix (Cat #1725274, Hercules, CA, USA), forward and reverse primers (as shown in Table 1), and nuclease-free H₂O (for the no template control to detect and exclude possible DNA contamination). Each run contained 7 standard curve points in duplicate. DNA amplification was done in, Bio-Rad CFX96 Touch (Hercules, CA, USA) under the following conditions: initial denaturation at 95° C. for 30 s, 40 cycles of denaturation at 95° C. for 15 s, various annealing temperatures according to Integrated DNA Technologies (IDT) for 30 s and elongation at 60° C. for 30 s. The data on the expression levels of the genes were obtained as Cp values based on the ‘second derivative maximum’ (automated method) as computed by the Bio-Rad CFX Maestro 1.1 Software (Version 4.1.2433.1219, Hercules, CA, USA). Gene expression was normalized to the expression of 18S. For each of the thirteen genes, the reactions were run in duplicate. All results were quantified by including a standard curve in the RT-qPCR analysis. The next four points of the standard curve were prepared by a 1:10 dilution. Each point of the standard curve was included in duplicate. A graph of Cp vs. log 10 concentrations was produced by the software and the efficiencies were calculated as 10 (1/slope). The specificity of the amplified RT-qPCR products was verified by melting curve analysis (60-95° C.) after 40 cycles, which resulted in different specific products, each with a specific melting temperature. RT-qPCR efficiency (E) values for the thirteen genes were as follows: DMT-1, 0.998; DcytB, 1.046; Ferroportin, 1.109; 18S rRNA, 0.994, Δ-6 Desaturase, 0.925; ZnT7, 0.916; ZIP6, 0.961; Hepcidin, 0.976; NK-βκ1, 1.113; TNF-α, 1.046; AP, 1.015; SI, 1.032; Na+/K+ ATPase, 1.024.

### 2.5 Collection of Microbial Samples and Intestinal Contents DNA Isolation

The contents of the duodenum were placed into a sterile 15 mL tube (Corning, Corning, NY, USA) containing 9 mL of sterile 1X phosphate buffered saline (PBS) and homogenized by vortexing with glass beads (3 mm diameter) for 3 minutes. Debris was removed by centrifugation at 1000 × g for 5 minutes, and the supernatant was collected and centrifuged at 4000 × g for 20 minutes. The pellet was washed twice with 1X PBS and stored at -80° C. until DNA extraction.

To extract DNA, the pellet was re-suspended in 50 mM EDTA and treated with 10 mg/mL lysozyme (Sigma Aldrich CO., St. Louis, MO) for 45 min at 37° C. The bacterial genomic DNA was then isolated using a Wizard Genomic DNA purification kit (Promega Corp., Madison, WI) following the manufacturer’s instructions.

### 2.6 PCR Amplification of Bacterial 16 s rDNA

Primers for Lactobacillus, Bifidobacterium, L. Plantarum, Clostridium, and E. coli were designed according to previously published data [20,21]. The universal primers, which identify all known strains of bacteria in the intestine, were prepared with the invariant region in the 16S rRNA of bacteria and used as internal standard to normalize the results. PCR products were separated by electrophoresis on 2 wt. % agarose gel, stained with ethidium bromide, and quantified using the Quantity One 1-D analysis software (BioRad, Hercules, CA, USA).

### 2.7. 16S rRNA Gene Amplification, Sequencing and Analysis

16S rRNA gene amplification, sequencing, and analysis was performed as previously described [22]. Microbial genomic DNA was extracted from cecal samples using the PowerSoil DNA isolation kit, as described by the manufacturer (MoBio Laboratories Ltd., Carlsbad, CA, USA). Bacterial 16S rRNA gene sequences were PCR-amplified from each sample using the 515F-806R primers for the V4 hypervariable region of the 16S rRNA gene, including 12-base barcodes. The complete methodology is indicated in the supplementary materials.

### 2.8 Glycogen Analysis

Glycogen analysis was obtained from pectoralis muscle as previously described [7,23]. Briefly, the frozen pectoral muscle samples were homogenized in 8 wt. % perchloric acid. Samples were then centrifuged at 12,000 × g for 15 minutes. The supernatant was removed, and 1.0 mL of petroleum ether was added to each tube. After mixing, the petroleum ether fraction was removed and samples from the bottom layer were transferred to a 96-well plate containing 300 µL of color reagent. All samples were read at a wavelength of 450 nm in an ELISA reader and the amount of glycogen was calculated according to a standard curve. The amount of glycogen present in pectoral sample was determined by multiplying the weight of the tissue by the amount of glycogen per 1 g of wet tissue.

### 2.9 Tissue Morphology Examination

Intestinal morphology examination was performed as was previously described [19]. Duodenum samples were fixed in fresh 4 wt. % (v/v) buffered formaldehyde, dehydrated, cleared, and embedded in paraffin. Serial sections were cut at 5 µm and placed on glass slides. Intestinal sections were deparaffinized in xylene, rehydrated in a graded alcohol series, stained with Alcian Blue/Periodic acid-Schiff, and examined by light microscopy. The following variables were measured in the intestine: villus height, villus width, crypt depth, Paneth cell number per crypt, Paneth cell width, goblet cell number, goblet cell diameter, types of goblet cells in the villi epithelium, and types of goblet cells within the crypts. Measurements in each segment were performed with a light microscope using CellSens Standard software (Olympus, Waltham, MA, USA).

### 2.10 Statistical Analysis

All values are expressed as means ± standard error, n = 6-12. Experimental treatments for the intraamniotic administration assay were arranged in a completely randomized design. The results were analyzed by one-way ANOVA. For significant p-values, a post-hoc Duncan test was used to compare test groups. Statistical analysis was carried out using SPSS software (version 20.0, IBM, Armonk, NY, USA). The level of significance was established at p < 0.05.

### 3. Results

### 3.1. Body Weight and Cecum Weight

The body weight of the 2.5 wt. % genistein group is significantly higher than the non-injected group (p < 0.05, Table 2). Among cecum weights, the no injection and H₂O groups demonstrate significantly greater values when compared to the 5 wt. % inulin and 2.5 wt. % genistein groups (p < 0.05).

Table 2. Body weight and cecum weight in all groups¹.

TABLE 2

Body weight and cecum weight in all groups ¹.

| | | |
|---|---|---|
| Treatment Group | Average Body Weight (g) | Average Cecum Weight (g) |
| No Injection | 43.23 ± 1.44 ^{b} | 0.60 ± 0.05^{a} |
| H₂O | 44.62 ± 1.43 ^{ab} | 0.59 ± 0.05 ^{a} |
| 5 wt. % Inulin | 46.04 ± 1.18 ^{ab} | 0.43 ± 0.05 ^{b} |
| 1.25 wt. % Genistein | 45.83 ± 0.99 ^{ab} | 0.50 ± 0.04 ^{ab} |
| 2.5 wt. % Genistein | 47.69 ± 1.30 ^{a} | 0.44 ± 0.03 ^{b} |

¹ Values are means ± SEM, n = 6. ^{a,b} Treatment groups not indicated by the same letter are significantly different (p < 0.05).

### 3.2. Hemoglobin and Glycogen Concentrations

Blood hemoglobin (Hb) levels in the 1.25 wt. % genistein group are significantly elevated compared to the No Injection, H₂O, and 5 wt. % inulin groups (p < 0.05, Table 3). The blood hemoglobin of the 2.5 wt. % genistein group is higher than the No Injection group and significantly higher versus the H₂O and 5 wt. % inulin groups. Among average glycogen, there was no significant difference between the genistein treated and no injection groups (p > 0.05).

TABLE 3

Blood hemoglobin (Hb) concentrations (g/dL) and pectoral muscle glycogen concentrations (mg/g) ¹

| | | |
|---|---|---|
| Treatment Group | Average Hb (g/dL) | Average Glycogen (mg/g) |
| No Injection | 10.10 ± 2.40 ^{bc} | 0.019 1 0.005 ^{a} |
| H₂O | 9.68 ± 2.50 ^{c} | 0.014 ± 0.003 ^{a} |
| 5 wt. % Inulin | 9.56 ± 0.92 ^{c} | 0.002 ± 0.001 ^{b} |
| 1.25 wt. % Genistein | 14.98 ± 0.45 ^{a} | 0.008 ± 0.003 ^{ab} |
| 2.5 wt. % Genistein | 14.23 ± 0.79 ^{ab} | 0.015 ± 0.004 ^{a} |

¹ Values are the means ± SEM, n=6-12. ^{a-c} Treatment groups not indicated by the same letter are significantly different (p < 0.05).

### 3.3. Gene Expression of Fe, Zn, BBM Functionality, and Pro-Inflammatory Related Proteins

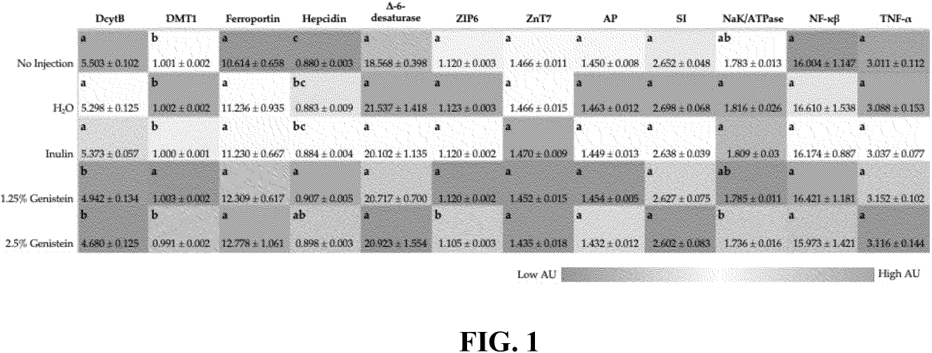

FIG. 1 shows the effect of the intraamniotic administration of experimental solutions on intestinal and liver (hepcidin) gene expression. Values are the means ± SEM, n = 6. ^{a-c} Per gene, treatments groups not indicated by the same letter are significantly different (p < 0.05). DcytB, Duodenal cytochrome b; DMT1, Divalent metal transporter 1; ZIP6, Zinc Transport Protein 6; ZnT7, Zinc transporter 7; AP, Amino peptidase; SI, Sucrose isomaltase; NaK/ATPase, Sodium, Potassium and Adenosine triphosphate; NF_{K}B1, Nuclear factor Kappa B Subunit 1; TNF-α, Tumor necrosis factor-alpha.

### 3.3.1. Fe-Related Proteins

For the proteins responsible directly for Fe uptake, the gene expression of DMT1 is downregulated in the 1.25 wt. % genistein when compared to the no injection and H₂O controls (p < 0.05). DcytB was significantly downregulated (p < 0.05) in the genistein treatment groups when compared to the no injection, H₂O, and inulin groups. Hepcidin was significantly upregulated (p < 0.05) with genistein exposure when compared to the no injection group. There were no significant differences in ferroportin expression between groups.

### 3.3.2. Zn-Related Proteins

For the proteins responsible directly for Zn uptake, ZIP6 was significantly downregulated (p < 0.05) in the 2.5 wt. % genistein group when compared to all other treatment groups. There were no significant differences in ZnT7 or Δ-6-desaturase expression between groups.

### 3.3.3. Inflammatory Cytokines and BBM Functionality

There were no significant differences in gene expression of aminopeptidase (AP), sucrose isomaltase (SI), sodium, potassium and adenosine triphosphate (NaK/ATPase) when comparing the treatment groups to the no injection group. There were no significant differences in gene expression of nuclear transcription factor (NF-ĸβ) and tumor necrosis factor (TNF-α) between groups.

### 3.4 Morphometric Analysis of Duodenal Villi, Depth of Crypts, Goblet Cells, and Paneth Cells

TABLE 4

Effects of intraamniotic administration of experimental genistein solutions on duodenal small intestinal villus¹.

| | | | |
|---|---|---|---|
| Treatment Group | Villus Height (µm) | Villus Width (µm) | Villus Surface Area (mm2) |
| No Injection | 201.18 ± 4.94 ^{b} | 33.73 ± 0.67 ^{e} | 112.51 ± 4.28 ^{d} |
| H₂O | 204.74 ± 4.52 ^{b} | 41.92 ± 1.01 ^{d} | 143.33 ± 5.27 ^{c} |
| 5 wt. % Inulin | 246.64 ± 5.14 ^{a} | 50.98 ± 1.03 ^{a} | 206.92 ± 6.37 ^{a} |
| 1.25 wt. % Genistein | 204.18 ± 3.73 ^{b} | 44.51 ± 0.86 ^{c} | 146.97 ± 4.55 ^{c} |
| 2.5 wt. % Genistein | 238.22 ± 3.17 ^{a} | 48.27 ± 0.87 ^{b} | 184.13 ± 4.66 ^{b} |

¹ Values are the means + SEM, n = 5. ^{a-c} Treatment groups not indicated by the same letter are significantly different (p < 0.05).

The villus height, width, and surface area of the 2.5 wt. % genistein were significantly increased (p < 0.05) when compared with the no injection and H₂O groups. The 1.25 wt. % genistein group had significantly (p < 0.05) greater villus width in comparison with the no injection and H₂O groups. The 2.5 wt. % genistein group had significantly higher (p < 0.05) villus height, width and surface area compared to the 1.25 wt. % genistein.

TABLE 5

Effects of intraamniotic administration of experimental genistein solutions on villi goblet cells¹.

| Treatment Group | Villi Goblet Cell Diameter (µm) | Villi Goblet Cell Number (Unit) | | | |
|---|---|---|---|---|---|
| | | Acidic | Neutral | Mixture | Total |
| No Injection | 2.86 ± 0.02 ^{d} | 13.59 ± 0.39 ^{d} | 0.01 ± 0.01 ^{c} | 3.50 ± 0.23 ^{c} | 17.09 ± 0.49 ^{d} |
| H₂O | 3.11 ± 0.03 ^{c} | 15.03 ± 0.39 ^{c} | 0.01 ± 0.01 ^{c} | 5.76 ± 0.30 ^{b} | 20.80 ± 0.47 ^{c} |
| 5 wt. % Inulin | 2.74 ± 0.03 ^{e} | 16.39 ± 0.54 ^{c} | 0.10 ± 0.02 ^{b} | 6.53 ± 0.30 ^{a} | 23.02 ± 0.60 ^{b} |
| 1.25 wt. % Genistein | 3.41 ± 0.03 ^{b} | 23.49 ± 0.67 ^{a} | 0.09 ± 0.03 ^{b} | 1.69 ± 0.13 ^{e} | 25.26 ± 0.67 ^{a} |
| 2.5 wt. % Genistein | 3.50 ± 0.03 ^{a} | 22.01 ± 0.51 ^{b} | 0.19 ± 0.04 ^{a} | 2.70 ± 0.16 ^{d} | 24.89 ± 0.54 ^{a} |

¹ Values are the means ± SEM, n = 5. ^{a-e} Treatment groups not indicated by the same letter are significantly different (p < 0.05).

The villi goblet cell diameter and total number of goblet cells were significantly higher (p < 0.05) in the genistein treatment groups than the no injection, H₂O, and inulin groups. More specifically, the acidic and neutral villi goblet cell count of both treatment groups were significantly higher (p < 0.05) than the control groups, and the mixture villi goblet cells were significantly reduced (p < 0.05) when compared with the controls.

TABLE 6

Effects of intraamniotic administration of experimental genistein solutions on crypt goblet cells¹.

| Treatment Group | Crypt Goblet Cell Diameter (µm) | Crypt Goblet Cell Number (Unit) | | | |
|---|---|---|---|---|---|
| | | Acidic | Neutral | Mixture | Total |
| No Injection | 2.68 ± 0.02 ^{b} | 5.46 ± 0.18 ^{d} | 0.00 ± 0.00 ^{a} | 1.49 ± 0.09 ^{b} | 6.95 ± 0.21 ^{d} |
| H₂O | 2.65 ± 0.02 ^{b} | 6.07 ± 0.18 ^{c} | 0.00 ± 0.00 ^{a} | 1.76 ± 0.08 ^{a} | 7.83 ± 0.19 ^{c} |
| 5 wt. % Inulin | 2.51 ± 0.02 ^{c} | 7.97 ± 0.16 ^{b} | 0.00 ± 0.00 ^{a} | 1.19 ± 0.08 ^{c} | 9.15 ± 0.16 ^{b} |
| 1.25 wt. % Genistein | 2.89 ± 0.02 ^{a} | 8.51 ± 0.14 ^{a} | 0.00 ± 0.00 ^{a} | 0.74 ± 0.06 ^{d} | 9.25 ± 0.14 ^{ab} |
| 2.5 wt. % Genistein | 2.63 ± 0.02 ^{b} | 8.81 ± 0.19 ^{a} | 0.00 ± 0.00 ^{a} | 0.88 ± 0.06 ^{d} | 9.68 ± 0.19 ^{a} |

¹ Values are the means ± SEM, n = 5. ^{a-d} Treatment groups not indicated by the same letter are significantly different (p < 0.05).

The crypt goblet cell diameter of the 1.25 wt. % genistein group was significantly greater (p < 0.05) than all control groups. The 2.5 wt. % genistein group had a significantly higher diameter compared to the 5 wt. % inulin group. Genistein exposure resulted in a significantly higher (p < 0.05) total crypt goblet cell count when compared with the no injection and H₂O groups. More specifically, the acidic crypt goblet cell count of both genistein treatment groups was significantly higher (p < 0.05) when compared with the no injection, H₂O, and inulin groups. Genistein exposure resulted in significantly reduced mixed crypt goblet cells (p < 0.05) when compared with the no injection, H₂O, and inulin groups.

Table 7. Effects of intraamniotic administration of experimental genistein solutions on crypt depth and Paneth cells ¹.

TABLE 7

Effects of intraamniotic administration of experimental genistein solutions on crypt depth and Paneth cells ¹.

| | | | |
|---|---|---|---|
| Treatment Group | Crypt Depth (µm) | # Crypt Paneth Cells | Crypt Paneth cell Diameter (µm) |
| No Injection | 22.45 ± 0.39 ^{d} | 1.48 ± 0.05 ^{d} | 1.67 ± 0.03 ^{b} |
| H₂O | 39.07 ± 0.80 ^{a} | 2.46 ± 0.11 ^{c} | 1.82 ± 0.04 ^{a} |
| 5 wt. % Inulin | 35.00 ± 0.43 ^{b} | 2.56 ± 0.09 ^{c} | 1.68 ± 0.03 ^{b} |
| 1.25 wt. % Genistein | 26.36 ± 0.46 ^{c} | 2.92 ± 0.10 ^{b} | 1.78 ± 0.04 ^{a} |
| 2.5 wt. % Genistein | 23.65 ± 0.46 ^{d} | 3.24 ± 0.11 ^{a} | 1.65 ± 0.03 ^{b} |

¹ Values are the means ± SEM, n = 5. ^{a-d} Treatment groups not indicated by the same letter are significantly different (p < 0.05).

The number of crypt Paneth cells was significantly greater (p < 0.05) for the genistein treatment groups compared to the no injection, H₂O, and inulin groups. The crypt depth for the treatment groups was significantly lower (p < 0.05) compared to the H₂O-injection group. The 1.25 wt. % genistein group had a significantly (p < 0.05) higher crypt Paneth cell diameter compared to the no injection and 5 wt. % inulin groups.

### 3.5. Intestinal Content Bacterial Expression

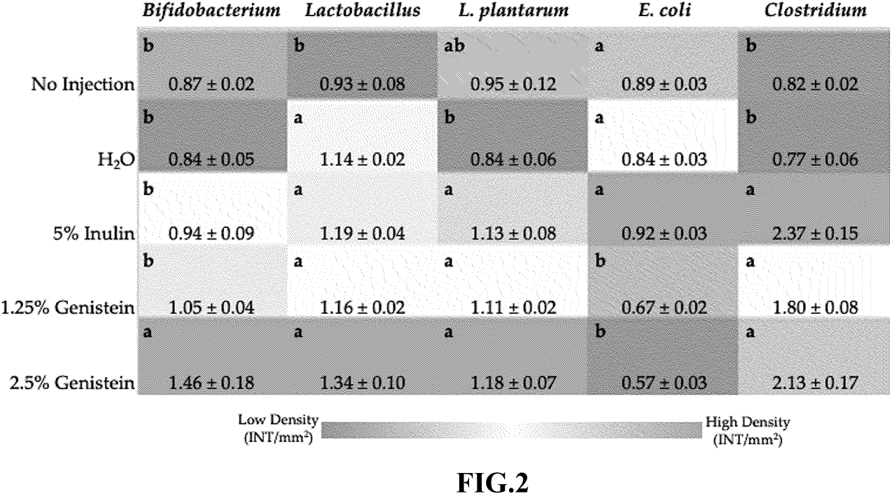

FIG. 2 shows the effects of intra-amniotic injections on duodenal genera and species-level bacterial populations (day of hatch). Values are means ± SEM, n = 5. ^{a,b} Per bacterial category, treatments groups that do not share any letters are significantly different (p < 0.05). FIG. 2 shows the chick duodenal genera and species-level bacterial populations. The relative abundance of Bifidobacterium spp., which is considered a probiotic bacteria, was significantly increased (p < 0.05) with 2.5 wt. % genistein exposure when compared with all other treatment groups. Lactobacillus spp. relative abundance was significantly increased (p < 0.05) with genistein exposure when compared with the no injection control. L. plantarum, a probiotic bacteria associated with increased Fe absorption, was significantly increased (p < 0.05) in the genistein exposed groups and 5 wt. % inulin control when compared with the H₂O injected control. Exposure to genistein resulted in significantly decreased (p < 0.05) relative abundance of E. coli when compared with all other treatment groups. Clostridium spp. relative abundance was significantly increased (p < 0.05) in the genistein treated groups and 5 wt. % inulin control when compared with the no injection and H₂O injection controls.

### 4. Discussion

In the present Example, Applicant evaluated the effect of intraamniotic genistein administration on mineral status, duodenal brush border membrane development and functionality, as well as intestinal microbiota. Although the ingestion of genistein has been associated with marked physiological changes associated with cancer and metabolic syndrome, further understanding into the tissue-level effects of genistein ingestion is needed [6,24,25]. Presently, there does not yet exist studies in literature which directly measure the effects of genistein on the combination of mineral status, BBM morphology or functionality, and intestinal microbiota.

The intraamniotic administration of genistein had a positive effect on intestinal development, as demonstrated by increased enterocyte proliferation. Duodenal morphometric analysis demonstrated a significant (p < 0.05) dose responsive effect of genistein treatment on increasing villus surface area versus the no injection control, indicative of improved digestive enzyme and absorptive capacity [7]. A significantly (p < 0.05) reduced crypt depth was observed with genistein administration when compared to the H₂O injection control group, which has been shown to be a marker of efficient tissue turnover and good condition of the gut [26]. The increase in villus surface area and reduction in crypt depth are in accordance with other genistein administration trials using the in vivo chicken model [27,28]. Additionally, increased proliferation in total villi and crypt goblet cells and increase in the proportion of villi acidic and crypt acidic (p < 0.05) goblet cells were observed when compared to the no injection and H₂O injection controls. This is an indication of increased synthesis and secretion of acidic luminal mucin by duodenal goblet cells [11,12]. In addition to serving as a protective intestinal epithelial barrier, this mucin also functions a habitat that supports probiotic populations and promotes epithelial cell function [29]. Taken as a whole, this demonstrates that the intraamniotic administration of genistein can modulate brush border membrane development and functionality.

The Gallus gallus model harbors a complex and active intestinal microbiota, significantly and directly influenced by host genetics, environment, and diet [20,30]. There is a significant resemblance at the phylum level between the gut microbiota of Gallus gallus and humans, with Bacteroidetes, Firmicutes, Proteobacteria, and Actinobacteria representing the dominant bacterial phyla in both [31]. Soy isoflavone treatment has previously been shown to alter intestinal bacterial populations in vivo, including increases in populations of SCFA-producing bacteria [1,24,32]. In the duodenum, the relative abundance of Bifidobacterium spp., which is considered a probiotic bacteria species, was significantly increased with 2.5 wt. % genistein exposure when compared with all other treatment groups. Lactobacillus spp. relative abundance was significantly increased with genistein exposure when compared with the no injection control. Further, Linear discriminant analysis Effect Size (LefSe) analysis found that genistein treatment enriched pathways associated with de novo synthesis of vitamin B₁₂ (FIG. S1), where bacteria from the Lactobacillus genus represent a small number of bacteria known to encode the complete de novo biosynthetic pathway of vitamin B₁₂ [33,34]. L. plantarum, a probiotic bacteria associated with increased Fe absorption, was significantly increased in the genistein exposed groups and 5 wt. % inulin control when compared with the H₂O injected control [35]. L. Plantarum produces glucosidases that can hydrolyze isoflavones (glycosides) into metabolites (aglycones) with increased antioxidant activity [36]. Clostridium spp. was significantly increased in the genistein treated groups, and butyrate-producing (SCFA) bacteria, such as Roseburia and E. hallii, from Clostridium cluster XIVa have previously been observed to be increased with genistein exposure in vitro [4]. The increase in Lactobacillus spp., Bifidobacterium spp., and Clostridium spp. abundance, may further contribute, directly or indirectly, to the increased bioavailability of Fe and Zn, as these bacterial genera produce SCFAs, which reduce the intestinal pH, and therefore, may increase mineral (as Fe and Zn) solubility and therefore absorption [7,37,38].

Our previous research suggested soy isoflavone (daidzein) intraamniotic administration has the potential to improve dietary Fe bioavailability [16]. In our current study, BBM gene expression analysis demonstrated that genistein downregulated DMT1 (transports Fe²⁺ into duodenal enterocyte) and DcytB (reduces Fe³⁺ to Fe²⁺) and upregulated ferroportin (transports Fe²⁺ into blood) and hepcidin (binds to ferroportin, causes ferroportin internalization and degradation), relative to the control group, though these results were not necessarily dose-dependent or significant. In Fe sufficient or excess scenarios, based on protein functionalities, it is expected that DcytB, DMT1, and ferroportin would be downregulated, while hepcidin would be upregulated [39-44]. Though upregulation of ferroportin has previously been associated with Fe deficiency, genistein treatment was found to upregulate ferroportin expression in glial cells through estrogen receptor-β-dependent p38 MAPK activation, independent of Fe status [9,44]. Additionally, genistein administration has been shown to upregulate hepcidin expression, which directly influences ferroportin expression, in in vivo and in vitro liver cell models [10]. Blood Hb levels were increased with genistein administration when compared with the controls, which taken together with Fe-gene expression analysis, may generally indicate Fe status was improved by genistein administration. Genistein exposure resulted in ZIP6 (imports zinc across cell membrane) downregulation in comparison with the no injection control, potentially indicative of improved zinc status with genistein administration [45,46], or could be associated with estrogenic effects of soy isoflavone, where ZIP6 expression was found to be modulated with anti-estrogen treatment in breast cells [47,48]. Improvements in mineral status was potentially due to increased production of SCFA and increased proportion of acidic goblet cells, which reduced the intestinal pH and led to increased mineral solubility, thus improving absorption [49].

Increases in body weight were observed in a dose-dependent manner when compared with the controls, with the 2.5 wt. % genistein treatment group observed to be significantly higher (p < 0.05) when compared to the no injection control. Given the short exposure time, a significant increase in body weight is unexpected, but when taken with improved Fe status and BBM development, and given that the in vivo Gallus gallus model is sensitive to dietary Fe and Zn deficiencies [38,50], a significant increase in body weight confirms the positive development effects related to genistein exposure [51]. Additional studies are warranted to assess shifts in mineral status, intestinal functionality and development, and intestinal microbiota post hatch and during a longer feeding trial associated with genistein consumption.

### 5. Summary of Example 1-+

The present Example at least demonstrates intraamniotic administration of genistein improved brush border membrane functionality through increases in villus architecture, surface area, and goblet cell expansion and related mucin production. Additionally, increases in relative abundance of bacterial populations associated with SCFA production were found. Consequently, these contributed to relative expression of various duodenal mineral and hepatic Fe proteins responsible for mineral absorption and transport associated improved Fe status. Given these findings, genistein represents a promising plant bioactive and should be further evaluated in both long-term animal and controlled human efficacy trials.

### References Related to Example 1

1. Lopez, P.; Sanchez, M.; Perez-Cruz, C.; Velazquez-Villegas, L.A.; Syeda, T.; Aguilar-Lopez, M.; Rocha-Viggiano, A.K.; Del Carmen Silva-Lucero, M.; Torre-Villalvazo, I.; Noriega, L.G., et al. Long-Term Genistein Consumption Modifies Gut Microbiota, Improving Glucose Metabolism, Metabolic Endotoxemia, and Cognitive Function in Mice Fed a High-Fat Diet. Mol Nutr Food Res 2018, 62, e1800313, doi:10.1002/mnfr.201800313.

2. Spagnuolo, C.; Russo, G.L.; Orhan, I.E.; Habtemariam, S.; Daglia, M.; Sureda, A.; Nabavi, S.F.; Devi, K.P.; Loizzo, M.R.; Tundis, R., et al. Genistein and cancer: current status, challenges, and future directions. Adv Nutr 2015, 6, 408-419, doi:10.3945/an.114.008052.

3. Setchell, K. Phytoestrogens: the biochemistry, physiology, and implications for human health of soy isoflavones. Am J Clin Nutr 1998, 68, 1333S-1346S.

4. Guadamuro, L.; Dohrmann, A.B.; Tebbe, C.C.; Mayo, B.; Delgado, S. Bacterial communities and metabolic activity of faecal cultures from equol producer and non-producer menopausal women under treatment with soy isoflavones. BMC Microbiol 2017, 17, 93, doi:10.1186/s12866-017-1001-y.

5. Matthies, A.; Blaut, M.; Braune, A. Isolation of a human intestinal bacterium capable of daidzein and genistein conversion. Appl Environ Microbiol 2009, 75, 1740-1744, doi:10.1128/AEM.01795-08.

6. Zhou, L.; Xiao, X.; Zhang, Q.; Zheng, J.; Li, M.; Wang, X.; Deng, M.; Zhai, X.; Liu, J. Gut microbiota might be a crucial factor in deciphering the metabolic benefits of perinatal genistein consumption in dams and adult female offspring. Food Funct 2019, 10, 4505-4521, doi:10.1039/c9fo01046g.

7. Carboni, J.; Reed, S.; Kolba, N.; Eshel, A.; Koren, O.; Tako, E. Alterations in the Intestinal Morphology, Gut Microbiota, and Trace Mineral Status Following Intra-Amniotic Administration (Gallus gallus) of Teff (Eragrostis tef) Seed Extracts. Nutrients 2020, 12, doi:10.3390/nu12103020.

8. Dias, D.M.; Costa, N.M.B.; Nutti, M.R.; Tako, E.; Martino, H.S.D. Advantages and limitations of in vitro and in vivo methods of iron and zinc bioavailability evaluation in the assessment of biofortification program effectiveness. Crit RevFood Sci Nutr 2018, 58, 2136-2146, doi:10.1080/10408398.2017.1306484.

9. Persichini, T.; Maio, N.; di Patti, M.C.; Rizzo, G.; Colasanti, M.; Musci, G. Genistein up-regulates the iron efflux system in glial cells. Neurosci Lett 2010, 470, 145-149, doi:10.1016/j.neulet.2009.12.074.

10. Zhen, A.W.; Nguyen, N.H.; Gibert, Y.; Motola, S.; Buckett, P.; Wessling-Resnick, M.; Fraenkel, E.; Fraenkel, P.G. The small molecule, genistein, increases hepcidin expression in human hepatocytes. Hepatology 2013, 58, 1315-1325, doi:10.1002/hep.26490.

11. Hou, T.; Tako, E. The In Ovo Feeding Administration (Gallus Gallus)-An Emerging In Vivo Approach to Assess Bioactive Compounds with Potential Nutritional Benefits. Nutrients 2018, 10, doi:10.3390/nu10040418.

12. Dias, D.M.; Kolba, N.; Hart, J.J.; Ma, M.; Sha, S.T.; Lakshmanan, N.; Nutti, M.R.; Martino, H.S.D.; Glahn, R.P.; Tako, E. Soluble extracts from carioca beans (Phaseolus vulgaris L.) affect the gut microbiota and iron related brush border membrane protein expression in vivo (Gallus gallus). Food Res Int2019, 123, 172-180, doi:10.1016/j.foodres.2019.04.060.

13. Mahler, G.J.; Esch, M.B.; Tako, E.; Southard, T.L.; Archer, S.D.; Glahn, R.P.; Shuler, M.L. Oral exposure to polystyrene nanoparticles affects iron absorption. Nat Nanotechnol 2012, 7, 264-271, doi:10.1038/nnano.2012.3.

14. Consortium, I.C.G.S. Sequence and comparative analysis of the chicken genome provide unique perspectives on vertebrate evolution. Nature 2004, 432, 695-777.

15. Warkentin, T.; Kolba, N.; Tako, E. Low Phytate Peas (Pisum sativum L.) Improve Iron Status, Gut Microbiome, and Brush Border Membrane Functionality In Vivo (Gallus gallus). Nutrients 2020, 12, doi:10.3390/nu12092563.

16. Hartono, K.; Reed, S.; Ankrah, N.A.; Glahn, R.P.; Tako, E. Alterations in gut microflora populations and brush border functionality following intra-amniotic daidzein administration. RSC Advances 2015, 5, 6407-6412, doi:10.1039/c4ra10962g.

17. Poschner, S.; Maier-Salamon, A.; Zehl, M.; Wackerlig, J.; Dobusch, D.; Pachmann, B.; Sterlini, K.L.; Jager, W. The Impacts of Genistein and Daidzein on Estrogen Conjugations in Human Breast Cancer Cells: A Targeted Metabolomics Approach. Front Pharmacol 2017, 8, 699, doi:10.3389/fphar.2017.00699.

18. Decuypere, E.; Michels, H. Incubation temperature as a management tool: a review. World’s Poultry Science Association 1992, 48, 28-38.

19. Pereira da Silva, B.; Kolba, N.; Stampini Duarte Martino, H.; Hart, J.; Tako, E. Soluble Extracts from Chia Seed (Salvia hispanica L.) Affect Brush Border Membrane Functionality, Morphology and Intestinal Bacterial Populations In Vivo (Gallus gallus). Nutrients 2019, 11, doi:10.3390/nu11102457.

20. Zhu, X.Y.; Zhong, T.; Pandya, Y.; Joerger, R.D. 16S rRNA-based analysis of microbiota from the cecum of broiler chickens. Appl Environ Microbiol 2002, 68, 124-137, doi:10.1128/aem.68.1.124-137.2002.

21. Gorsuch, J.; LeSaint, D.; VanderKelen, J.; Buckman, D.; Kitts, C.L. A comparison of methods for enumerating bacteria in direct fed microbials for animal feed. J Microbiol Methods 2019, 160, 124-129, doi:10.1016/j.mimet.2019.04.003.

22. Reed, S.; Neuman, H.; Glahn, R.P.; Koren, O.; Tako, E. Characterizing the gut (Gallus gallus) microbiota following the consumption of an iron biofortified Rwandan cream seeded carioca (Phaseolus Vulgaris L.) bean-based diet. PLoS One 2017, 12, e0182431, doi:10.1371/journal.pone.0182431.

23. Dreiling, C.; Brown, D.; Casale, L.; Kelly, L. Muscle Glycogen: Comparison of Iodine Binding and Enzyme Digestion Assays and Application to Meat Samples. Meat Science 1987, 20, 167-177.

24. Huang, G.; Xu, J.; Lefever, D.E.; Glenn, T.C.; Nagy, T.; Guo, T.L. Genistein prevention of hyperglycemia and improvement of glucose tolerance in adult non-obese diabetic mice are associated with alterations of gut microbiome and immune homeostasis. Toxicol Appl Pharmacol 2017, 332, 138-148, doi:10.1016/j.taap.2017.04.009.

25. Guevara-Cruz, M.; Godinez-Salas, E.T.; Sanchez-Tapia, M.; Torres-Villalobos, G.; Pichardo-Ontiveros, E.; Guizar-Heredia, R.; Arteaga-Sanchez, L.; Gamba, G.; Mojica-Espinosa, R.; Schcolnik-Cabrera, A., et al. Genistein stimulates insulin sensitivity through gut microbiota reshaping and skeletal muscle AMPK activation in obese subjects. BMJ Open Diabetes Res Care 2020, 8, doi:10.1136/bmjdrc-2019-000948.

26. Sobolewska, A.; Bogucka, J.; Dankowiakowska, A.; Elminowska-Wenda, G.; Stadnicka, K.; Bednarczyk, M. The impact of synbiotic administration through in ovo technology on the microstructure of a broiler chicken small intestine tissue on the 1(st) and 42(nd) day of rearing. J Anim Sci Biotechnol 2017, 8, 61, doi:10.1186/s40104-017-0193-1.

27. Kamboh, A.A.; Zhu, W.Y. Individual and combined effects of genistein and hesperidin on immunity and intestinal morphometry in lipopolysacharide-challenged broiler chickens. Poult Sci 2014, 93, 2175-2183, doi:10.3382/ps.2014-03971.

28. Glisic, M.; Boskovic, M.; Baltic, M.Z.; Sefer, D.; Radovanovic, A.; Djordjevic, V.; Raseta, M.; Markovic, R. Performance, intestinal histomorphology and bone composition of broiler chickens fed diets supplemented with genistein. South African Journal of Animal Science 2020, 50, 241-252, doi:10.4314/sajas.v50i2.7.

29. Engevik, M.A.; Luk, B.; Chang-Graham, A.L.; Hall, A.; Herrmann, B.; Ruan, W.; Endres, B.T.; Shi, Z.; Garey, K.W.; Hyser, J.M., et al. Bifidobacterium dentium Fortifies the Intestinal Mucus Layer via Autophagy and Calcium Signaling Pathways. mBio 2019, 10, doi:10.1128/mBio.01087-19.

30. Yegani, M.; Korver, D.R. Factors affecting intestinal health in poultry. Poult Sci 2008, 87, 2052-2063, doi:10.3382/ps.2008-00091.

31. Qin, J.; Li, R.; Raes, J.; Arumugam, M.; Burgdorf, K.S.; Manichanh, C.; Nielsen, T.; Pons, N.; Levenez, F.; Yamada, T., et al. A human gut microbial gene catalogue established by metagenomic sequencing. Nature 2010, 464, 59-65, doi:10.1038/nature08821.

32. Huang, G.; Xu, J.; Cai, D.; Chen, S.Y.; Nagy, T.; Guo, T.L. Exacerbation of Type 1 Diabetes in Perinatally Genistein Exposed Female Non-Obese Diabetic (NOD) Mouse Is Associated With Alterations of Gut Microbiota and Immune Homeostasis. Toxicol Sci 2018, 165, 291-301, doi:10.1093/toxsci/kfy162.

33. Torres, A.C.; Vannini, V.; Bonacina, J.; Font, G.; Saavedra, L.; Taranto, M.P. Cobalamin production by Lactobacillus coryniformis: biochemical identification of the synthetized corrinoid and genomic analysis of the biosynthetic cluster. BMC Microbiol 2016, 16, 240, doi:10.1186/s12866-016-0854-9.

34. De Angelis, M.; Bottacini, F.; Fosso, B.; Kelleher, P.; Calasso, M.; Di Cagno, R.; Ventura, M.; Picardi, E.; van Sinderen, D.; Gobbetti, M. Lactobacillus rossiae, a vitamin B12 producer, represents a metabolically versatile species within the Genus Lactobacillus. PLoS One 2014, 9, e107232, doi:10.1371/journal.pone.0107232.

35. Axling, U.; Onning, G.; Martinsson Niskanen, T.; Larsson, N.; Hansson, S.R.; Hulthen, L. The effect of Lactiplantibacillus plantarum 299v together with a low dose of iron on iron status in healthy pregnant women: A randomized clinical trial. Acta Obstet Gynecol Scand 2021, 10.1111/aogs.14153, doi:10.1111/aogs.14153.

36. Landete, J.M.; Curiel, J.A.; Rodríguez, H.; de las Rivas, B.; Muñoz, R. Aryl glycosidases from Lactobacillus plantarum increase antioxidant activity of phenolic compounds. Journal of Functional Foods 2014, 7, 322-329, doi:10.1016/j.jff.2014.01.028.

37. Pacifici, S.; Song, J.; Zhang, C.; Wang, Q.; Glahn, R.P.; Kolba, N.; Tako, E. Intra Amniotic Administration of Raffinose and Stachyose Affects the Intestinal Brush Border Functionality and Alters Gut Microflora Populations. Nutrients 2017, 9, doi:10.3390/nu9030304.

38. Tako, E.; Glahn, R.P.; Knez, M.; Stangoulis, J.C.R. The effect of wheat prebiotics on the gut bacterial population and iron status of iron deficient broiler chickens. Nutrition Journal 2014, 13.

39. Imam, M.U.; Zhang, S.; Ma, J.; Wang, H.; Wang, F. Antioxidants Mediate Both Iron Homeostasis and Oxidative Stress. Nutrients 2017, 9, doi:10.3390/nu9070671.

40. Lane, D.J.; Bae, D.H.; Merlot, A.M.; Sahni, S.; Richardson, D.R. Duodenal cytochrome b (DCYTB) in iron metabolism: an update on function and regulation. Nutrients 2015, 7, 2274-2296, doi:10.3390/nu7042274.

41. Gulec, S.; Anderson, G.J.; Collins, J.F. Mechanistic and regulatory aspects of intestinal iron absorption. Am J Physiol Gastrointest Liver Physiol 2014, 307, G397-409, doi:10.1152/ajpgi.00348.2013.

42. Fuqua, B.K.; Vulpe, C.D.; Anderson, G.J. Intestinal iron absorption. J Trace Elem Med Biol 2012, 26, 115-119, doi:10.1016/j.jtemb.2012.03.015.

43. Morgan, E.H.; Oates, P.S. Mechanisms and regulation of intestinal iron absorption. Blood Cells MolDis 2002, 29, 384-399, doi:10.1006/bcmd.2002.0578.

44. Zimmermann, M.B.; Hurrell, R.F. Nutritional iron deficiency. The Lancet 2007, 370, 511-520, doi:10.1016/s0140-6736(07)61235-5.

45. Maares, M.; Haase, H. A Guide to Human Zinc Absorption: General Overview and Recent Advances of In Vitro Intestinal Models. Nutrients 2020, 12, doi:10.3390/nu12030762.

46. Takagishi, T.; Hara, T.; Fukada, T. Recent Advances in the Role of SLC39A/ZIP Zinc Transporters In Vivo. Int J MolSci 2017, 18, doi:10.3390/ijms18122708.

47. Takatani-Nakase, T. Zinc Transporters and the Progression of Breast Cancers. Biol. Pharm. Bull. 2018, 41, 1517-1522.

48. Lopez, V.; Kelleher, S.L. Zip6-attenuation promotes epithelial-to-mesenchymal transition in ductal breast tumor (T47D) cells. Exp Cell Res 2010, 316, 366-375, doi:10.1016/j.yexcr.2009.10.011.

49. Wang, X.; Kolba, N.; Liang, J.; Tako, E. Alterations in gut microflora populations and brush border functionality following intra-amniotic administration (Gallus gallus) of wheat bran prebiotic extracts. Food Funct 2019, 10, 4834-4843, doi:10.1039/c9fo00836e.

50. Reed, S.; Qin, X.; Ran-Ressler, R.; Brenna, J.T.; Glahn, R.P.; Tako, E. Dietary zinc deficiency affects blood linoleic acid: dihomo-gamma-linolenic acid (LA:DGLA) ratio; a sensitive physiological marker of zinc status in vivo (Gallus gallus). Nutrients 2014, 6, 1164-1180, doi:10.3390/nu6031164.

51. Tako, E.; Glahn, R.P. Iron Status of the Late Term Broiler (Gallus gallus) Embryo and Hatchling. International Journal of Poultry Science 2011, 10, 42-48.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of (a) preventing intestinal abnormality, dysbiosis, malabsorption, malnutrition, infection, and/or disease in the avian, (b) modifying an intestinal microbiome structure of an avian, or both (a) and (b), the method comprising:
administering, to an avian, an amount of genistein or a genistein formulation, wherein the amount of genistein or genistein formulation is effective to
a. modify the intestinal microbiome structure of the avian;
b. increase a nutrient and/or mineral bioavailability and/or improve nutrient and/or mineral status in the avian;
c. increase body weight gain of the avian;
d. decrease feed conversion ratio of the avian;
e. support normal avian intestinal health and/or functionality and/or improve intestinal health and/or functionality, microbiome structure, intestinal health or functionality expression signature, and any combination thereof;
f. correct an iron or Zn deficiency in the avian;
g. modify expression of one or more of the following genes in the avian: DMT1, Ferroportin, DcytB, Bepcidin, Δ-6-desaturase, ZIP6, ZnT7, NF-ĸβ, TNF-α, AP, SI, NaK/ATPase, and 18S;
h. to preventing intestinal abnormality, dysbiosis, malabsorption, malnutrition, infection, and/or disease in the avian or
i. any combination of (a)-(h).

2. The method of claim 1, wherein the amount of genistein administered ranges from about 0.1 wt. % to 5 wt. %, of a dietary supplement, of a feed supplement, of a total diet, or of a feed formulation or is otherwise administered at an amount such that the total amount of genistein in a total diet of the avian ranges from about 0.1 wt. % to 5 wt. %.

3. The method of claim 2, wherein the amount of genistein administered ranges from about 0.1 wt. % to 1 wt. %, 1.25 wt. % to about 2.5 wt. %, or about 1 wt. % to about 5 wt. % of the genistein formulation, of a dietary supplement, of a feed supplement, of a total diet, or of a feed formulation or is otherwise administered at an amount such that the total amount of genistein in a total diet of the avian ranges from about 0.1-1 wt. %, 1.25 wt. % to about 2.5 wt. %, or about 1 wt. % to about 5 wt. %.

4. The method of claim 1, wherein the avian is a developing embryo, neonate, a young avian, an adolescent avian, or a mature adult avian.

5. The method of claim 1, wherein administration is during embryonic development, optionally directly to a developing embryo or indirectly to a sexually mature avian.

6. The method of claim 1, wherein administration is via in ovo injection, intraamniotic injection, or oral.

7. The method of claim 1, wherein the avian, is an avian of the genus Gallus, Melegris, Dromaius, Struthio, Phoenicopterus, Callipepla, Cyrtonyx, Dactylortyx, Philortyx, Colinus, Odontophorus, Oreortyx, Rhynchortyx, Coturnix, Synoicus, Perdicula, or Ophrysia.

8. The method of claim 1, wherein modification of the microbiome structure comprises an increase in number of probiotic, SCFA producing bacteria, and/or non-pathogenic bacteria and/or decrease the number of pathogenic or non-probiotic bacterium.

9. The method of claim 8, wherein the (i) non-pathogenic bacteria comprises one or more Bifidobacterium, lactobacillus, L.plantarum, Clostridium bacteria; (ii) the pathogenic bacteria comprises E. coli; or (iii) both (i) and (ii).

10. The method of claim 1, wherein normal avian intestinal health and/or functionality and/or improve intestinal health and/or functionality is measured by intestinal morphology, microbiome structure, intestinal health or functionality expression signature or any combination thereof.

11. The method of claim 10, wherein measuring intestinal morphology comprises determining goblet cell number, crypt Paneth cell number, or both.

12. The method of claim 10, wherein an intestinal health or functionality expression signature comprises the expression of one or more intestinal nutrient transporters, one more intestinal mineral transporters, one or more markers of intestinal inflammation, one or more intestinal NaK/ATPase exchangers, one or more intestinal mineral regulators, one or more liver mineral regulators, or any combination thereof.

13. A formulation comprising:
an amount of genistein optionally effective, in an avian, to
a. modify the intestinal microbiome structure of the avian;
b. increase a nutrient and/or mineral bioavailability and/or improve nutrient and/or mineral status in the avian;
c. increase body weight gain of the avian;
d. decrease feed conversion ratio of the avian;
e. support normal avian intestinal health and/or functionality and/or improve intestinal health and/or functionality, microbiome structure, intestinal health or functionality expression signature, and any combination thereof;
f. correct an iron or Zn deficiency in the avian;
g. modify expression of one or more of the following genes in the avian: DMT1, Ferroportin, DcytB, Bepcidin, Δ-6-desaturase, ZIP6, ZnT7, NF-ĸβ, TNF-α, AP, SI, NaK/ATPase, and 18S;
h. to preventing intestinal abnormality, dysbiosis, malabsorption, malnutrition, infection, and/or disease in the avian or
i. any combination of (a)-(h).

14. The formulation of claim 13, wherein the amount of genistein ranges from about 0.1 wt. % to 5 wt. % of the formulation or is included in the formulation at an amount such that the amount of genistein in a total diet of a subject to which it is administered ranges from about 0.1 wt. % to 5 wt. %.

15. The formulation of claim 14, wherein the amount of genistein ranges from about 0.1-1 wt. %, 1.25 wt. % to about 2.5 wt. %, or about 1 wt. % to about 5 wt. % of the formulation or is included in the formulation at an amount such that the amount of genistein in a total diet of a subject to which it is administered ranges from 0.1-1 wt. %, 1.25 wt. % to about 2.5 wt. %, or about 1 wt. % to about 5 wt. %.

16. The formulation of claim 13, wherein the formulation is a feed formulation or dietary supplement.

17. The formulation claim 13, wherein at least part or all of the genistein is from soybeans included in the formulation.

18. The formulation claim 13, wherein the avian is a neonate, a young non-avian, an adolescent avian, or a mature adult avian.

19. The formulation of claim 13, wherein the subject is an avian of the genus Gallus, Melegris, Dromaius, Struthio, Phoenicopterus, Callipepla, Cyrtonyx, Dactylortyx, Philortyx, Colinus, Odontophorus, Oreortyx, Rhynchortyx, Coturnix, Synoicus, Perdicula, or Ophrysia.

20. The formulation of claim 13, wherein the formulation is adapted for in ovo administration, intraamniotic administration, or oral administration.
